(19) United States
(12) Patent Application Publication
(10) Pub. No.: US 2018/0296112 A1
(43) Pub. Date: Oct. 18, 2018

(54) METHODS AND APPARATUSES FOR SEIZURE MONITORING

(71) Applicant: Brain Sentinel, Inc., San Antonio, TX (US)

(72) Inventors: Michael R. Girouard, Shavano Park, TX (US); Damon P. Cardenas, San Antonio, TX (US); Luke E. Whitmire, San Antonio, TX (US)

(21) Appl. No.: 15/953,293

(22) Filed: Apr. 13, 2018

Related U.S. Application Data
(60) Provisional application No. 62/485,268, filed on Apr. 13, 2017.

(51) Int. Cl. A61B 5/04; A61B 5/0488; A61B 5/00; A61B 5/11

(52) U.S. Cl. CPC A61B 5/04012; A61B 5/0488; A61B 5/4094; A61B 5/1118; A61B 5/0402; A61B 5/746; A61B 5/0022; A61B 2560/0238; A61B 2560/0209; A61B 5/7264

(57) ABSTRACT

Methods and systems are described for detecting and classifying seizure-related events. In some embodiments, the methods and systems herein may include adjustment of one or more threshold settings used for seizure detection in order to improve sensitivity and/or battery performance of a mobile EMG detection unit.

### CROSS REFERENCE TO RELATED APPLICATIONS

This application claims priority to U.S. Provisional Patent Application No. 62/485,268 filed Apr. 13, 2017. The disclosure of the foregoing application is herein fully incorporated by reference.

### FIELD

This application relates generally to systems and methods for monitoring a patient for seizure activity using electromyography and calibration thereof.

### BACKGROUND

A seizure may be characterized as abnormal or excessive synchronous activity in the brain. At the beginning of a seizure, neurons in the brain may begin to fire at a particular location. As the seizure progresses, this firing of neurons may spread across the brain, and in some cases, many areas of the brain may become engulfed in this activity. Seizure activity in the brain may cause the brain to send electrical signals to activate different muscles of the body.

Techniques designed for studying and monitoring seizures have typically relied upon electroencephalography (EEG), which characterizes electrical signals using electrodes attached to the scalp or head region of a seizure-prone individual or seizure patient. In EEG, electrodes may be positioned so as to measure such activity; that is, electrical activity originating from neuronal tissue. Alternatively, electromyography (EMG) may be used for seizure detection. In EMG, an electrode may be placed on or near the skin, over a muscle, to detect electrical activity resulting from muscle activation.

Detecting an epileptic seizure using EEG typically requires attaching many electrodes and associated wires to the head and using amplifiers to monitor brainwave activity. The multiple EEG electrodes may be very cumbersome and generally require some technical expertise to apply and monitor. Furthermore, confirming a seizure may require observation in an environment provided with video monitors and video recording equipment. Unless used in a staffed clinical environment, such equipment may not be intended to determine if a seizure is in progress, but rather to provide a historical record of the seizure after the incident. Such equipment is usually meant for hospital-like environments where a video camera recording or caregiver's observation may provide corroboration of the seizure and is typically used as part of a more intensive care regimen such as a hospital stay for patients who experience multiple seizures. Upon discharge from the hospital, a patient may be sent home, often with little further monitoring.

Some ambulatory devices for diagnosis of seizures may also be based on EEG, but because of the above shortcomings those devices are not designed or suitable for long-term home use or daily wearability. Other seizure alerting systems may operate by detecting motion of the body, usually the extremities. Such systems may generally operate on the assumption that while suffering a seizure, a person will move erratically and violently. For example, accelerometers may be used to detect violent extremity movements. However, depending upon the type of seizure, this assumption may or may not be true. Electrical signals sent from the brain during some seizures may be transmitted to many muscles simultaneously, which may result in muscles fighting each other and effectively canceling out violent movement. In other words, the muscles may work to make the person rigid rather than cause actual violent movement. Thus, some seizures may not be consistently detected with accelerometer-based detectors.

In addition, some ambulatory devices for diagnosis of seizures are generally not suited to grade seizures based on intensity and/or duration, nor are they suited to differentiate seizure-related signals based on type. Or, some devices may only classify seizures using a plurality of sensor types, a configuration which may significantly limit useful battery life and/or impact cost and comfort of use. For example, in some detection systems, different types of seizures may often be grouped together. Accordingly, ambulatory devices for seizure detection may be ill-suited to customize responses for different types of detected seizure-related events. However, some detected seizures may not pose a significant risk of adverse effects of having a seizure. Nevertheless, if detection is made without further classification, unnecessary, and cost-prohibitive signaling of alarms in response to non-threatening events may be the only way to also respond to potentially dangerous events. Thus, some ambulatory devices may not be ideally suited for cost-effective monitoring of some patients. Moreover, such devices may be ill-equipped to timely provide data that may help caregivers provide an appropriate care response. For example, some caregiver decisions may ideally be influenced by how long a seizure was active or how long one or more parts of a seizure were active. However, unless timely classification of seizure related data is made, and the results provided, caregivers may not be privy to such information when such is needed. Also, when using some ambulatory devices, caregivers may misdiagnose some conditions, including some that may benefit from condition-specific therapies. Systems and methods for characterization of data collected using ambulatory devices and for generating statistical information useful to caregivers are thus noticeably deficient or missing.

Moreover, some ambulatory devices may operate with high sensitivity for seizure detection and may assert to be applicable without patient specific calibration. However, some of those systems may, at least for some patients, operate with poor selectivity for detection seizures. That is, rates of false positive detections may be significant. Specific calibration of devices may generally improve selectivity for seizure detection and reduce rates of false positive detection. However, where such calibration is executed in a controlled setting, such as a hospital, such approaches may be inconvenient and/or costly for the patient. Other calibration procedures may demand that a patient execute one or more daily operations. However, such approaches may be inconvenient for the patient and may not always be effective, particularly for patients who may have limited physical or mental capacity necessary for properly executing such procedures. In addition, some calibration routines may not be designed to consider effects of control power consumption in devices. Notably, such deficiencies may limit applicability of detection strategies that may include a combination of EMG detection routines and which may be applied in ambulatory detection devices.

Accordingly, there is a need for improved seizure detection methods and apparatuses that address the above problems and that can be used in non-institutional or institutional environments without many of the cumbersome electrodes to the head or extremities. For example, there is a need for detection methods that are suited to analyze seizures by type and/or intensity in order to customize alarm responses and to better characterize seizure events to help medically and surgically manage patient care. There is further a need for improved methods for calibrating EMG detection devices. For example, there is a need for calibration methods that may be automatically executed without significantly affecting device performance, including power consumption.

### SUMMARY

In some embodiments, EMG detection systems herein may be configured for automatic calibration of threshold settings for monitoring a patient for detection of seizure activity. The EMG detection system may include a wireless EMG detection unit (32), the wireless detection unit including one or more EMG electrodes (54), wherein the EMG electrodes may be configured to collect an EMG signal for a patient. The wireless detection unit (32) may further be configured for remote communication with one or more caregiver devices (42, 44). The EMG detection systems may further include an identification module (92), the identification module including a processor configured to execute a first group of one or more seizure-detection routines for determining one or more property values of the EMG signal and comparing the one or more property values to one or more initial thresholds for detection of one or more seizure-related events. The EMG detection systems may further include a classification module (94), wherein the classification module may include a processor configured to execute a second group of one or more seizure-detection routines for classifying individual ones among the one or more seizure-related events as being associated with one or more physiological activity types in order to provide classification data, the one or more physiological activity types including a generalized tonic-clonic seizure type and at least one other physiological activity type. The EMG detection systems may further include a threshold adjustment module (96), wherein the threshold adjustment module includes a processor configured to access the classification data and use the classification data to evaluate one or more performance metrics for the one or more seizure-detection routines when applying the one or more initial thresholds. In some embodiments, the one or more performance metrics may include a sensitivity for detection of generalized tonic-clonic seizures and a selectivity for detection of generalized tonic-clonic seizure; wherein the threshold adjustment module is further configured to automatically adjust the one or more initial thresholds based on said one or more performance metrics in order to calibrate the EMG detection unit.

In some embodiments, methods are described herein for calibrating an EMG system for monitoring a patient for seizure activity. The methods may include disposing an EMG detection unit including one or more EMG electrodes in association with one or more patient muscles, the one or more EMG electrodes configured for collecting an EMG signal in a form substantially representing seizure-related muscle activity; collecting the EMG signal using the one or more EMG electrodes; processing the EMG signal using a first group of one or more seizure-detection routines, the one or more seizure-detection routines configured for determining one or more property values of the EMG signal and comparing the one or more property values to one or more initial threshold in order to detect one or more seizure-related events. The methods may further include classifying the one or more seizure-related events using a second group of one or more additional seizure-detection routines, the one or more additional seizure-detection routines configured to determine how individual seizure-related events relate to one or more physiological activity types, the one or more physiological activity types including a generalized tonic-clonic seizure type and at least one other physiological activity type. The methods may further include evaluating how well said first group of one or more seizure-detection routines functions in detecting said one or more seizure-related events based on one or more performance metrics for the one or more seizure-detection routines when the one or more seizure-detection routines apply the one or more initial thresholds, the one or more performance metrics including a sensitivity for detection of generalized tonic-clonic seizures and a selectivity for detection of generalized tonic-clonic seizure; and updating the one or more initial thresholds based on the evaluation of the one or more performance metrics in order to calibrate the EMG detection unit.

In some embodiments, EMG detection systems are described herein for monitoring of a patient for detection of seizure activity. The EMG detection systems may include a wireless EMG detection unit, the wireless detection unit including one or more EMG electrodes, the one or more EMG electrodes configured to collect an EMG signal for a patient substantially continuously over time; wherein the detection unit is configured for remote communication with one or more caregiver devices. The EMG detection systems may further include an identification module including a processor configured to execute a first group of one or more seizure-detection routines for determining one or more property values of the EMG signal and comparing the one or more property values to one or more initial thresholds for detection of one or more seizure-related events; wherein the identification module is further configured to initiate execution of a classification module based on the detection of the one or more seizure-related events. The EMG detection systems may further include a classification module including a processor configured to selectively execute a second group of one or more seizure-detection routines for classifying individual ones among said one or more seizure-related events as being associated with one or more physiological activity types, the one or more physiological activity types including a generalized tonic-clonic seizure type and at least one other physiological activity type. The EMG detection systems may further include an alarm initiation module including a processor configured to send one or more alarms to the one or more caregiver devices in response to detection of the one or more seizure-related events.

In some embodiments, methods are described herein for monitoring a patient for seizure activity. The methods may include monitoring a patient using one or more EMG electrodes to obtain an EMG signal; processing the EMG signal to determine if the patient may be experiencing one or more seizure-related events the processing including executing at least one of a first group of one or more first seizure-detection routines, the one or more first seizure-detection routines include instructions for calculating one or more property values of the EMG signal and comparing the one or more property values to one or more thresholds in detection of the one or more seizure-related events. The methods may further include executing one or more second seizure-detection routines; wherein the one or more second seizure-detection routines include instructions for classifying individual ones among the one or more seizure-related events in order to obtain classified seizure-related event data; wherein the classified seizure-related event data include an identification of a relationship of the individual seizure-related events to one or more physiological activity types; wherein the one or more physiological activity types include a generalized tonic-clonic seizure type and at least one other physiological activity type, the at least one other physiological activity type selected from a psychogenic non-epileptic seizure type, a non-seizure movement type, and a physiological activity type that includes a combination of both the psychogenic non-epileptic seizure type and the non-seizure movement type. The method may further include evaluating one or more performance metrics for the one or more seizure-detection routines when using the one or more thresholds with respect to at least one of the one or more physiological activity types; and adjusting the one or more thresholds based on the one or more performance metrics.

In some embodiments, methods are described herein for monitoring a patient for seizure activity. The methods may include monitoring the patient using one or more EMG electrodes to obtain an EMG signal; processing, with a processor, the EMG signal to determine if the patient may be experiencing a seizure-related event; wherein said processing includes executing at least one of a first group of one or more seizure-detection routines; wherein said one or more seizure-detection routines include instructions for calculating one or more property values for said EMG signal and comparing said one or more property values to one or more thresholds in detection of said seizure-related event. The methods may further include executing one or more other seizure-detection routines; wherein said one or more other seizure-detection routines include instructions for classifying said seizure-related event as being associated with one or more physiological activity types; wherein said one or more physiological activity types include an epileptic seizure activity type and at least one other physiological activity type; and executing an emergency alarm if said seizure-related event is classified as being of said epileptic seizure activity type.

In some embodiments, methods are described herein for monitoring a patient for seizure activity. The methods may include monitoring said patient using one or more EMG electrodes to obtain an EMG signal; processing, with a processor, said EMG signal to determine if said patient may be experiencing one or more seizure-related events; wherein said processing includes executing at least one of a first group of one or more seizure-detection routines said one or more seizure-detection routines including instructions for calculating one or more property values of said EMG signal and comparing said one or more property values to one or more thresholds in detection of said one or more seizure-related events; initiating execution of one or more other seizure-detection routines if said processing indicates that said patient has experienced at least one of said one or more seizure-related events, said one or more other seizure-detection routines including instructions for classifying individual ones among said one or more seizure-related events in order to create classified seizure-related event data; and initiating one or more alarms if said classified seizure-related event data indicates that said patient has experienced a seizure.

In some embodiments, EMG detection systems are described herein for automatic calibration of threshold settings for monitoring a patient for detection of seizure activity. The EMG detection systems may include a wireless EMG detection unit (32), said wireless EMG detection unit including one or more EMG electrodes (54), the one or more EMG electrodes configured to collect an EMG signal from a patient, the wireless EMG detection unit (32) configured to remotely communicate with one or more caregiver devices (42, 44). The EMG detection systems may further include an identification module (92), said identification module including a processor configured to execute a first group of one or more first seizure-detection routines for determining one or more property values of said EMG signal and comparing said one or more property values to one or more initial thresholds for detection of one or more seizure-related events. The EMG detection systems may further a classification module (94), said classification module including a processor configured to execute a second group of one or more second seizure-detection routines for classifying individual seizure-related events as being associated with one or more physiological activity types, the one or more physiological activity types including a generalized tonic-clonic seizure type and a non-seizure activity type; and a threshold adjustment module (96), said threshold adjustment module including a processor configured to automatically adjust at least one of said one or more initial thresholds if a threshold number of said one or more seizure-related events are detected and classified as a non-seizure activity type.

In some embodiments, systems and methods herein may be configured to monitor a patient during one or more sessions wherein one or more earlier used or initial threshold settings used for detection of seizure-related muscle activity may be applied. In some embodiments, initial threshold settings may be predetermined. For example, predetermined settings may be based on empirical data collected for a group of patients, including, for example, all recorded patients or all patients of some demographic. In some embodiments, one or more initial threshold settings may also be determined based on data collected for a specific patient, including, for example, by having the patient execute one or more maximum voluntary contractions. In some embodiments, the one or more earlier used or initial threshold settings for detection of seizure-related muscle activity may be automatically adjusted to adjusted threshold settings based on data collected while monitoring the patient. For example, transition between threshold settings may be accomplished automatically during one or more reference or training periods. Transitioning of threshold settings may be achieved without interfering with patient monitoring. For example, in some embodiments, adjustment of threshold settings may occur without patient notification. Alternatively, a patient may be notified when threshold settings have been adjusted or when adjustment of settings has calibrated the device to a desired level. In some embodiments, notification that a device has been calibrated may also include informing the patient that the device is now expected to achieve a desired or expected battery life during normal operation

### BRIEF DESCRIPTION OF THE DRAWINGS

FIG. 1A is a schematic diagram that illustrates embodiments of a method of seizure detection that may include more than one seizure-detection routine.

FIG. 1B is another schematic diagram that illustrates other embodiments of a method of seizure detection that may include more than one seizure-detection routine.

FIG. 2 is a schematic diagram that illustrates embodiments of a system for seizure detection.

FIG. 3 is a schematic diagram that illustrates embodiments of a detection unit.

FIG. 4 is a schematic diagram that illustrates embodiments of a base station.

FIG. 5 is a schematic description of a seizure detection system.

FIG. 6 is a flowchart that illustrates embodiments of a method of seizure detection.

FIG. 7 is a flowchart that illustrates other embodiments of a method of seizure detection.

FIG. 8 is a flowchart that illustrates embodiments of a method for classification of seizure-related events data.

FIG. 9 is a flowchart that illustrates embodiments of methods for scoring, evaluating, and selecting one or more detection conditions.

FIG. 10A is a table that illustrates model classified seizure-related event data.

FIG. 10B is a table that illustrates model seizure-related event data organized to show a detection condition used in the events detection.

FIG. 10C is a table that illustrates an exemplary group of generated detection conditions.

FIG. 10D is a table that illustrates an exemplary group of performance metrics as assigned to various detection conditions.

### DETAILED DESCRIPTION

The following terms as used herein should be understood to have the indicated meanings.

The term “battery life” means a time or expected time of operation between when a fully-charged battery, or unit powered by the battery, may begin operations for monitoring a patient and when the battery or associated unit should be recharged.

The term “computer program” means a list of instructions that may be executed by a computer to cause the computer to operate in a desired manner.

The term “detection condition” refers to one or more seizure-detection routines and one or more thresholds or groups of thresholds that may be used to detect seizure-related events. For example, an identification module may use one or more detection conditions to evaluate an EMG signal for the presence of seizure-related muscle activity and clinical episodes where such activity may be present. For example, an identification module may be configured to select or use a detection condition, such as, for example, a seizure-detection routine configured to determine a T-squared value and compare the T-squared value to a T-squared threshold value. And, in such example, if the T-squared threshold value is exceeded or exceeded over some duration threshold, the seizure-detection routine may determine that a seizure-related event was detected.

The term “EMG signal” as used herein means a representation of one or more signals collected using one or more EMG electrodes. An EMG signal may include a digital representation of a signal collected by one or more EMG electrodes.

“Having” means including but not limited to.

The term “module” or “system module” refers to a collection of software and/or hardware that individually or in combination may perform one or more functions in a method or part of a method that may be used to monitor a patient for seizure activity.

The term “seizure-detection routine” refers to a method or part of a method that may be used to monitor a patient for seizure-related muscle activity. A seizure-detection routine may be run individually in a strategy for monitoring a patient or may be run in combination with other seizure-detection routines or methods in an overall strategy for patient monitoring. For example, a processor may execute a seizure-detection routine configured to process an EMG signal in order to calculate one or more values of one or more measurable properties of the EMG signal and compare the one or more properties to one or more thresholds in order to detect one or more seizure-related events.

The term “seizure-related muscle activity” as used herein refers to muscle activity that exhibits a measurable property (which may be characterized by a property value) detectable using EMG that is increased or more prevalent during any of various types of epileptic seizures, seizures associated with a seizure disorder, psychogenic or non-epileptic seizures (PNES), or other seizures when compared to one or more levels of the property measured for a patient at rest and in a normal state. Included among measurable properties that may be increased or may become more prevalent during the aforementioned seizures are levels of overall muscle activity, coherence of muscle groups, levels of rhythmic or repetitive muscle activation, other properties associated with the aforementioned seizures, and combinations thereof While some measurable properties using EMG may be more prevalent when a seizure is occurring, those properties may also be present or elevated, at least to some degree, during some non-seizure activities. Accordingly, as used in this disclosure, measured or detected seizure-related muscle activity may or may not be indicative of an actual seizure or epileptic seizure.

The term “seizure-related event” as used herein means a clinical episode or event in which a patient exhibits seizure-related muscle activity. A seizure-related event may or may not be associated with an actual seizure or epileptic seizure.

Where a range of values is described, it should be understood that intervening values, unless the context clearly dictates otherwise, between the upper and lower limit of that range, and any other stated or intervening value in other stated ranges, may be used within embodiments herein.

The apparatuses and methods described herein may be used to detect seizures and timely alert caregivers of seizure-related events. The apparatuses may include sensors disposed on, near, or underneath the skin of a patient or attached to a patient's clothing and may be configured for measurement of muscle electrical activity using EMG. In some embodiments, apparatuses herein may include one or more processors suitable to receive an EMG or other sensor signal and process the signal to detect seizure-related muscle activity. Detection of seizures using EMG electrodes is further described in, for example, Applicant's U.S. Pat. Nos. 8,386,025; 8,983,591; 9,186,105; 9,439,595; 9,439,596; 9,603,573; and 9,833,185, Applicant's U.S. patent application Ser. Nos. 14/233,904; 14/407,249; 14/816,924; and 14/920,665, Applicant's International Applications PCT/US14/61783, PCT/US14/68246, PCT/US15/49859, PCT/DK12/50215, PCT/US16/28005, PCT/US16/55925, PCT/US17/28429 and PCT/US17/64377 Applicant's U.S. Provisional Patent Application Nos. 61/875,429, 61/894,793, 61/910,827, 61/969,660, 61/979,225, 62/001,302, 62/032,147, 62/050,054, 62/096,331 and 62/324,786, the disclosures of each of which are herein fully incorporated by reference.

In some embodiments, the methods and apparatuses herein may include identification of seizure-related muscle activity and initiation of one or more responses if an associated seizure-related event is detected. For example, some of the methods and apparatuses herein may be configured to initiate one or more warning alarms, emergency alarms, alarm updates, or combinations thereof. And, the methods and apparatuses herein may be used to coordinate actions of different caregivers.

In some embodiments, methods and apparatuses herein may include classifying detected seizure-related events. Classifying as used herein may refer to further characterization of seizure-related muscle activity after initial detection of muscle activity determined to be seizure-related muscle activity. Notably, in some embodiments herein, classification may be performed to various levels without demanding collection of a plurality of data streams involving different sensor types and may further be performed in a device ideally suited for daily wear without significantly compromising battery life. Classification of seizure-related events may include characterization of detected seizure-related events as being associated with one or more types of physiological activity. For example, a seizure-related event may be characterized as a generalized tonic-clonic (GTC) seizure or seizure including one or more parts of a GTC seizure, a PNES event, a complex-partial seizure, a muscle movement associated with activity other than a seizure, another type of physiological activity, or activity type of certain duration or amplitude.

In some embodiments, classification of data may be used to initiate or update one or more alarm responses. For example, an alarm initially executed in the form of a warning message to one or more caregivers may be updated to one associated with an emergency alarm protocol. Further by way of example, a warning protocol may be updated to an emergency protocol, such as may include an immediate response to provide care to a patient.

In some embodiments, classification of data may be used to update one or more threshold settings used for seizure identification in a mobile detection unit. For example, in some embodiments, systems and methods herein may be configured for automatic calibration to a patient's individual musculature. Furthermore, systems and methods herein may be calibrated to detect seizure activity or detect and classify seizure activity, operations that may be particularly challenging for some patients, such as patients that may be obese and whom may include significant amounts of adipose tissue. In some embodiments, systems and methods herein may be designed to perform one or more internal calibrations without significantly disrupting system performance. And, in some embodiments, a combination of seizure-detection routines may be calibrated for use in patient monitoring, the routines calibrated to detect or detect and classify seizure-related events without unduly impacting device battery performance.

In some embodiments, methods and apparatuses herein may be directed to improving power consumption in systems for mobile patient monitoring. For example, in some embodiments, detection of seizure-related events and classification of the detected events may be performed in distinct or separate steps included in a method for seizure detection. For example, initial detection of seizure-related events may be accomplished using a first group of one or more seizure-detection routines that may execute continuously or periodically at a high rate and that may initiate a near-instantaneous response if a seizure-related event is detected. Classification of seizure-related events may be performed using a second group of one or more seizure-detection routines. In some embodiments, at least some of the routines in the second group of one or more seizure-detection routines may execute selectively in response to one or more positive detections of seizure-related events in at least one of the first group of one or more seizure-detection routines. Thus, some classification routines may only execute selectively not continuously, and routines that demand significant computational and/or power resources may be effectively used in systems suitable for ambulatory patient monitoring.

More generally, classification of seizure-related events may be used for a number of purposes in the various embodiments described herein. For example, in some embodiments, classification of seizure-related events may be used to minimize false positive alarm initiation, update one or more alert or alarm responses, provide information on the inter-muscular coherence of signals from different muscle groups, provide other information to one or more caregivers, update threshold settings useful for identification or detection of seizure-related muscle activity, or used for any combination of the aforementioned purposes.

In some embodiments, detection of seizure-related events may include use of one or more seizure-detection routines configured to provide a near-instantaneous response following physiological manifestation of seizure-related muscle activity. For example, an alarm or other response may sometimes be initiated within several seconds or even within less than about one second following physiological manifestation of seizure-related muscle activity. In order to provide a near instantaneous response following physiological manifestation of seizure-related muscle activity, a seizure-detection routine may periodically and at a high rate compare one or more property values of an EMG signal to a threshold. For example, some seizure-detection routines may examine one or more short sections of an EMG signal for the presence of an elevated EMG signal amplitude. If one or more elevated values of EMG signal amplitude are detected that are above one or more thresholds, a response may be almost immediately initiated. Accordingly, temporal lag between onset of seizure-related muscle activity and detection thereof may be minimized. In addition, some of the seizure-detection routines described herein that may operate continuously and which may process relatively short durations of EMG signal during a detection interval may demand lower computational and battery resources than other seizure-detection routines, including, for example, seizure-detection routines that may process longer sections of an EMG signal and/or that may perform more computationally intensive processing on an EMG signal.

For example, seizure-detection routines that process relatively short segments of an EMG signal (e.g., less than about several seconds of data) in order to determine an amplitude value or some statistical values calculated therefrom, such as a T-squared statistical value or principal component value, may generally operate using limited computational resources and without drawing large amounts of energy from a battery or other source of energy, advantages which may be particularly beneficial in patient-worn or personal mobile detection devices where battery and computational or processing resources may be limited. Other seizure-detection routines that may operate continuously without drawing large amounts of energy from a battery or other source of energy include, for example, routines designed to determine a number of crossings, i.e. zero-crossings, between an amplitude of an EMG signal, such as a filtered EMG signal, and a threshold value. For example, some embodiments of seizure-detection routines suitable for use herein and which include use of T-squared statistical values or principal component values are further described in U.S. Pat. No. 9,186,105 and U.S. Pat. No. 9,439,596, each of which is commonly owned by Applicant and fully incorporated herein by reference. Some embodiments of seizure-detection routines suitable for use herein and which include zero crossings are further described in PCT/DK2012/050215 which is also commonly owned by Applicant and fully incorporated herein by reference.

In some embodiments, determination of a T-squared value may include processing an EMG signal collected for a time period by filtering to select a plurality of frequency bands. For example, an EMG frequency spectrum may be broken up into a number of frequency bands, such as three or more, and one or more characteristics of each frequency band, such as an EMG signal amplitude or power content of the band, may be determined. A measured characteristic for a frequency band may be normalized by its variance and covariance with respect to the characteristic as measured in other frequency bands and the resulting normalized values processed to determine a T-squared statistical value.

However, at least for some patients, when using some of the aforementioned seizure-detection routines, it may be challenging to accurately set thresholds or initial thresholds suitable for detecting all seizure activity while also preventing false alarms based on inadvertent detection of non-seizure activity. For example, ideal thresholds suitable for achieving high sensitivity detection of seizures while minimizing false detections may vary between some patients. Some embodiments of methods herein may set initial thresholds suitable to achieve high sensitivity detection of even weak seizure-related muscle activity, even if selection of such thresholds may limit selectivity for detection of actual seizure events. However, some of those embodiments may further include use of a combination of seizure-detection routines in methods that facilitate automatic selection or adjustment of thresholds. For example, automatic adjustment of thresholds may be achieved during one or more periods wherein a device may collect data suitable to adjust or optimize conditions for detection of seizures. In some embodiments, one or more calibration periods may be executed wherein thresholds are adjusted seamlessly during regular patient monitoring or in one or more calibration periods without significant disruption to the patient. Thus, the methods herein may provide a significant advance over other methods previously used. For example, in some embodiments, automatic adjustment or selection of thresholds may be used in combination with or in lieu of some operations which may be executed to adjust threshold settings based on a patient's individual musculature, such as performing one or more maximum voluntary contractions (MVC). Some of those embodiments may be particularly advantageous for use with some patients who may find it difficult to reproducibly follow instructions or where execution of dedicated operations during a training period may be difficult or inconvenient.

Some forms of physiological activity may manifest as relatively brief or intermittent periods of muscle activation. That muscle activation may generally be detected with highest sensitivity when processing intervals of data that are of similar duration to the duration periods where muscle activation is present. For example, it may be difficult to detect brief or sporadic muscle twitches when calculating an integration value from detected EMG signals, or other statistical value calculated therefrom, over time intervals that are much longer than timeframes of muscle activation. Accordingly, in some embodiments, seizure-detection routines may process EMG signals collected over relatively short detection intervals, including, for example, detection intervals of less than about 5 seconds, less than about 3 seconds or less than about 1 second. While some of those routines may be optimized for sensitivity of detection for seizure-related muscle activity that may be relatively transient, it may be difficult to discriminate events associated with some forms of transient muscle activity from some other events. For example, it may be difficult to fully characterize all seizure-related events that a patient with epilepsy or another seizure disorder may experience using only routines that include the aforementioned relatively limited detection intervals. Some methods herein may be useful for detection of such activity, because such activity may be identified without always initiating an emergency response when such a response may not be needed.

Some embodiments of the methods herein that use a combination of detection routines may facilitate high sensitivity detection of some seizure-related events, including, for example, some events that may manifest over short time scales, but also facilitate a more complete and accurate characterization of other seizure-related events. For example, seizure-related events that may manifest over extended time periods may be characterized using appropriately long detection intervals. For example, complicated seizure-related events, including, for example, some events where muscle activity may change over the course of different event parts, may be characterized. Some of the aforementioned events may be ideally characterized using algorithms involving pattern recognition, some wavelets techniques or other processing techniques that may be computationally intensive or otherwise ill-suited for continuous use in a personal mobile sensor or other sensor that may operate with limited battery and/or computational resources.

For example, in some embodiments, differentiation of GTC seizure events from some other types of similarly presenting events, such as PNES events, may include use of seizure-detection routines that may analyze trends and changes in a collected EMG signal over time periods of at least about 10 seconds or in some cases significantly longer time periods. Other seizure-detection routines suitable to classify some seizure-related events, some of which may involve pattern recognition and/or comparison of data to a plurality of stored waveforms, may analyze portions of EMG data that extend for as long as several minutes, such as about 2 minutes to about 5 minutes, for example.

To reduce lag between physiological manifestation of seizure-related muscle activity and its detection, some routines herein may include detection intervals that may be staggered. However, because some seizure-detection routines suitable to classify some seizure-related events may use prolonged detection intervals and involve significant computations, it may be difficult or impossible to run some of those seizure-detection routines continuously and/or using staggered detection intervals without placing significant demands on computational resources and/or battery resources. Some of the embodiments herein, including, for example, embodiments wherein one or more seizure-detection routines useful for classification of seizure-related events are selectively executed by other seizure-detection routines may address this limitation associated with use of classification routines individually.

In some embodiments, a first group of one or more seizure-detection routines may be configured to gate or trigger execution of a second group of one or more seizure-detection routines suitable for classification of one or more seizure-related events. Advantageously, in some embodiments wherein more than one seizure-detection routine is selectively executed in response to detection of activity using one or more other seizure-detection routines, different ones among the more than one selectively executed seizure-detection routines may analyze collected EMG data over intervals that are individually optimized for classification of different types of seizure-related events. Moreover, high sensitivity detection and classification of seizure-related events may be achieved without demanding battery resources that would severely limit applicability and/or cost in a remote detection device.

In some of the embodiments herein, any of various suitable classification routines may be used. Classification of seizures based on the presence of one or more phases of seizure activity has been previously described by Applicant. For example, in U.S. application Ser. No. 14/920,665, filed Oct. 22, 2015, which is commonly owned by Applicant, methods for characterizing seizures based on a combination of seizure-detection routines wherein at least one of the routines is selective for clonic-phase seizure activity are described. As further described therein, analysis of samples of EMG signal including elevated signal amplitude may be useful in distinguishing epileptic seizures from some other events such as PNES events.

Some of the methods described in U.S. application Ser. No. 14/920,665 may include detection of samples of EMG signal including elevated amplitude and may include techniques for qualification of samples which may be related to clonic-phase seizure activity. Those techniques may further be used to qualify samples related to different parts of the clonic phase. For example, samples may be identified and included in groups for qualification of samples through each of initial, middle, and later portions of the clonic phase. Once qualified, various sample statistics may be determined, tracked over time, and used to better understand a seizure. For example, statistics of samples determined to be qualified-clonic-phase bursts may be used to differentiate a detected seizure for a patient with epilepsy from other seizures resulting from conditions other than epilepsy. In some embodiments, a clonic-phase burst activity level may be determined and may include one or more of a burst count number, burst count rate, certainty-weighted burst value, other metric of burst activity, and combinations thereof. In some embodiments, a burst activity level may be used to determine if the clonic phase of a seizure is detected and/or if a detected seizure-related event is classified as including a clonic phase. For example, a burst activity level may be compared to a threshold activity level in order to identify the presence of the clonic phase of a seizure.

In international application PCT/US16/28005, methods for detecting samples of EMG signal including elevated signal amplitude over background and generating a statistical summary of detected samples are further described. Some of the embodiments therein are particularly suited for detection of PNES events and differentiating those events from epileptic seizures. For example, in some embodiments, a seizure-detection routine may include processing EMG signal data to identify if the EMG signal includes elevations in signal amplitude; if elevations in signal amplitude are present, then analyzing if times between the elevations in signal amplitude within a clonic window change in a manner that is typical of either a PNES event or an epileptic seizure. If the times between the elevations change in a manner that is typical of a PNES event, the signal data may be associated with a PNES event. If the times between the elevations change in a manner that is typical of an epileptic seizure event, the signal data may be associated with the clonic phase of a seizure or GTC seizure event. For example, in some embodiments, the slope of a trend line for times between elevated portions of samples versus a detected sample number of less than about 0.8 may be used to classify an event as a PNES event. In contrast, in some embodiments, a trend line slope of greater than about 1.5 may be used to classify an event as being associated with a seizure condition typical of a patient with epilepsy.

Some of the aforementioned seizure-detection routines may execute one or more algorithms to detect and qualify samples. Some of those routines may further involve operations such as searching for particular patterns of activity among a group of samples and/or include test group construction, operations that may demand added computational resources. Some of those routines may further track how qualified samples change across different portions of a clonic phase of a seizure and may sometimes operate using detection intervals of at least about 10 seconds, about 20 seconds, or in some cases substantially longer detection intervals may be used.

In International Application No. PCT/US2017/028429, filed Apr. 19, 2017 and commonly owned by Applicant, other methods useful for classifying seizures are described. For example, in some embodiments, wavelet analysis may be used to perform seizure semiology and to analyze whether an event may be associated with a GTC seizure or other type of seizure such as a non-seizure event, PNES event, or complex-partial seizure. In some embodiments, wavelet analysis routines may analyze data collected over intervals of between about 30 seconds to about 60 seconds. In some other embodiments, wavelet analysis routines may analyze data collected over intervals of between about 45 seconds to as long as about 5 minutes, for example.

In some embodiments, suitable methods of classifying seizures as described in either of U.S. application Ser. No. 14/920,665, U.S. Application No. 62/324,786, International Application PCT/US16/28005, or other applications referenced herein, may be used as one or more seizure-detection routines in an overall strategy of patient monitoring. Particularly, methods of classification of seizure events may, in some preferred embodiments, be selectively executed when gated by one or more seizure-detection routines that operate continuously or in a semi-continuous manner, such as at a high rate of repetition.

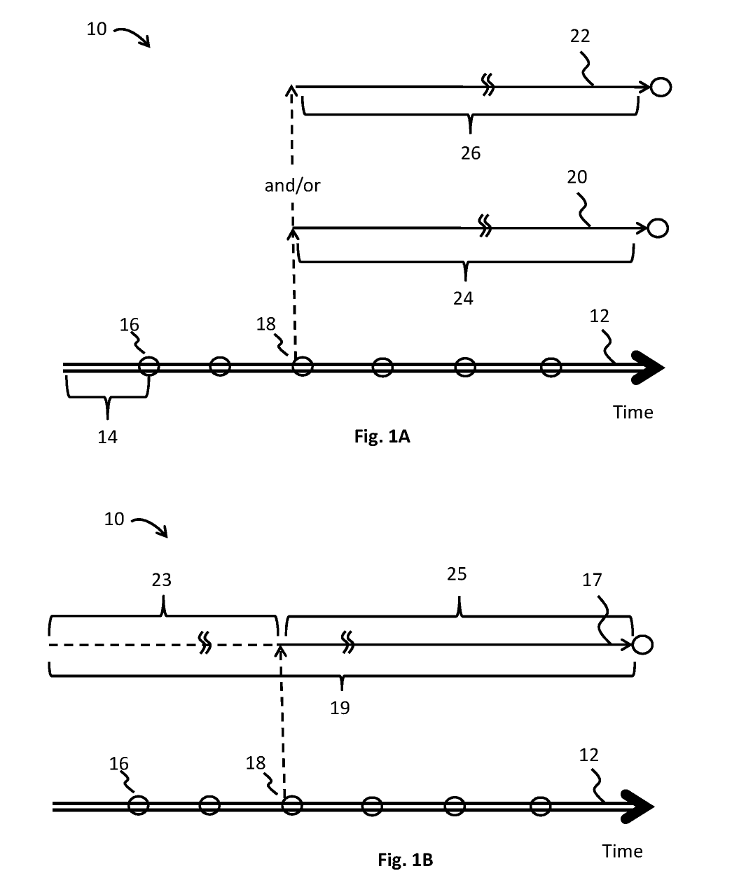

FIGS. 1A and 1B illustrate some embodiments of a method 10 for monitoring a patient for seizure activity which may include one or more seizure-detection routines. As shown therein, in some embodiments, a first seizure-detection routine (execution of which is illustrated by double line 12) may include collecting data within a series of detection intervals such as the detection interval 14. Following collection of data in a certain detection interval, a processor including instructions to execute the first seizure-detection routine may evaluate whether EMG data collected in that detection interval indicated the presence of seizure-related muscle activity. For example, as shown at time 16, it may be determined that during detection interval 14 a patient exhibited only normal muscle activity. In contrast, upon completion of another detection interval, the processor may determine that seizure-related muscle activity was detected. For example, as shown at time 18, using a first seizure-detection routine, it may be determined that seizure-related muscle activity was present. One or more responses may then be initiated.

In some embodiments of the method 10, a response to detection of seizure-related muscle activity using a first seizure-detection routine may include triggering execution of one or more other seizure-detection routines. For example, in FIG. 1A, positive detection of seizure-related muscle activity at time 18 is shown to trigger execution of one seizure-detection routine (execution of which is represented by line 20) and/or another seizure-detection routine (execution of which is represented by line 22). The seizure-detection routines (execution of which is shown by lines 20, 22) may analyze EMG signal collected in intervals (24, 26). In some embodiments, the intervals (24, 26), which may be of the same duration or of different durations, may be longer than duration interval 14. In addition, in some embodiments, any of various processing techniques including execution of one or more wavelet transforms, pattern recognition and/or test group construction, and/or other data intensive operations may be executed in either or both of the aforementioned seizure-detection routines (execution of which is shown by lines 20, 22).

FIG. 1B shows other embodiments of the method 10. As shown therein, positive detection of seizure-related muscle activity at time 18 is shown to trigger execution of the seizure-detection routine whose execution is represented by line 17. The seizure-detection routine whose execution is represented by line 17 may be characterized as having a detection interval 19. In some embodiments, at least a portion of detection interval 19 may be included in buffer interval 23 and a remaining interval of detection interval 19 may be included in remainder interval 25. In some of those embodiments, the buffer interval 23 may include collected data that is stored in accessible memory, and if a positive detection of seizure-related muscle activity is made (e.g., as shown at time 18), the data collected in buffer interval 23 may then be processed in the selectively executed seizure-detection routine whose execution is represented by line 17. For example, data collected in buffer interval 23 may be combined with data collected in remainder interval 25 in order to produce data in the detection interval 19. Data collected in detection interval 19 may then be evaluated for seizure activity. Thus, some of the detection intervals described herein (19, 24, 26) may include data from any of various data collected prior to, together with, or after a time 18 wherein a decision was made to execute a seizure-detection routine suitable for processing data in the detection interval. For example, the selection or orientation of a detection interval may be made to capture data suitable to classify a detected seizure-related event as a particular physiological activity type.

In some embodiments, one or more of the above routines (e.g., routines the execution of which is shown by lines 12, 17, 20, and 22) may include detection intervals that are staggered. For example, windows may be staggered to decrease lag between physiological manifestation of seizure-related muscle activity and one or more responses initiated based on a detection of that activity. However, a degree to which detection intervals may be staggered may be limited in order to reduce or minimize use of computational and/or battery resources. In some embodiments, following initiation of one or more selectively initiated routines (e.g., routines the execution of which is shown by lines 17, 20, and 22), the routines may operate for a predetermined time period, including, for example, a predetermined period suitable for collection of EMG signal used in some number of detection intervals. Alternatively, in some embodiments, the one or more selectively initiated routines (e.g., routines the execution of which is shown by lines 17, 20, and 22), may operate until a signal is provided to stop collection. For example, if only normal levels of non-seizure activity are present, the classification routine may be given a stop or termination signal.

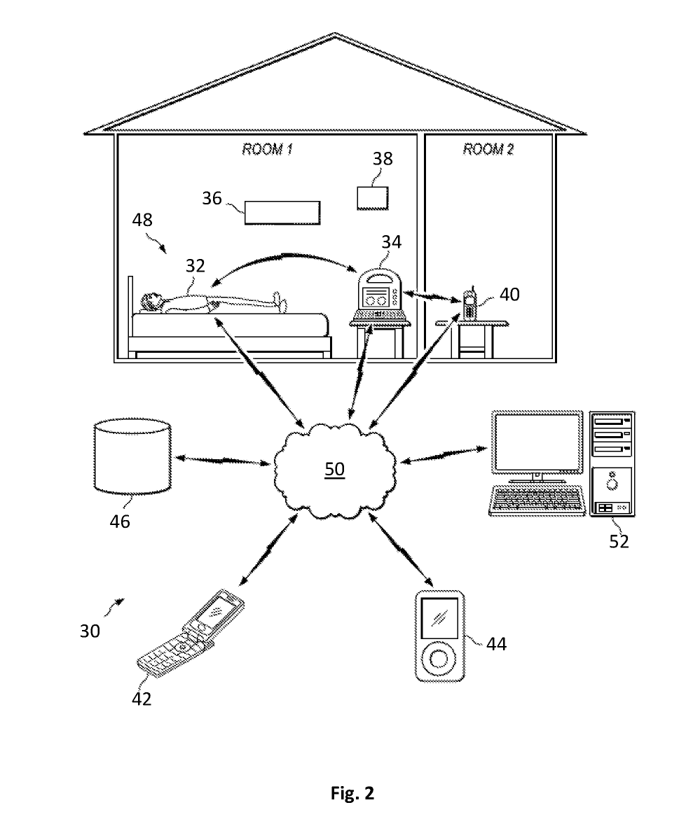

A variety of systems may be suitable for collecting EMG and other patient-related data, organizing such data for system optimization, and for initiating an alarm in response to detection of seizure-related muscle activity. FIG. 2 illustrates an exemplary embodiment of such a system that may be configured to monitor a patient for seizure activity using the methods described herein. In the embodiment of FIG. 2, a seizure detection system 30 may include a detection unit 32. The detection unit may be configured as a portable and wearable device disposed on or near (or even attached to) any suitable muscle or muscle groups that may be subject to motor manifestations during a seizure.

In some preferred embodiments, detection unit 32 may include surface EMG electrodes designed to be minimally intrusive and suitable for daily wear. For example, in some embodiments, EMG detection units herein may be wireless devices configured for repeated application to a patient's skin (e.g., applied daily or at some other suitable interval) using a suitable medical pressure sensitive adhesive. In some preferred embodiments, adhesives used herein may be biocompatible, hypoallergenic and may be applied to the skin without addition of solvent. For example, adhesives herein may comprise water-resistant materials, including, for example, biocompatible acrylic or silicone-based materials. In some embodiments, detection units may be light weight and further shaped to distribute and/or relieve stresses on the skin. For example, an adhesive patch may include one or more rounded protuberances designed to relieve skin stress.

In some embodiments, the system 30 may include any of various wireless local area network technologies. For example, a detection unit 32 may communicate wirelessly to the internet using WiFi, Bluetooth, or through another local network. And, using a local network a detection unit 32 may, in some embodiments, send data over the internet directly or via an intermediate base station 34. In some embodiments, a caregiver may be contacted directly through a local network such as WiFi. A base station 34 may be connected to the internet wirelessly (such as through a local network), or may be linked to the internet through a hard connection. And, in some embodiments, in addition to a detection unit 32 or in addition to a detection unit 32 and base station 34, a system 30 may, for example, include any of an acoustic sensor 36, a video camera 38, alert transceiver 40, or combination of the aforementioned elements.

The detection unit 32 may comprise one or more EMG electrodes capable of detecting electrical signals from muscles at or near the skin surface of a patient and delivering those electrical EMG signals to a processor for processing. The base station 34 may comprise a computer capable of receiving and processing EMG signals from the detection unit 32, acoustic data from acoustic sensor 36, and/or data from other sensors, determining from the processed signals whether a seizure may have occurred, and sending an alert to a caregiver. An alert transceiver 40 may be carried by, or placed near, a caregiver to receive and relay alerts transmitted by the base station 34 or to the internet. Other components that may be included in the system 30, including for example, wireless communication devices 42, 44, storage database 46, electronic devices for detecting changes in the integrity of an electrode skin interface, and one or more environmental transceivers.

In using the apparatus of FIG. 2, a patient 48 susceptible to epileptic or other seizures may be resting in bed, or may be at some other location as daily living may include, and may have a detection unit 32 in physical contact with or in proximity to his or her body. The detection unit 32 may be a wireless device so that the patient 48 may be able to get up and walk around without having to be tethered to an immobile power source or to a bulkier base station 34. For example, the detection unit 32 may be woven into a shirt sleeve or may be mounted to an armband or bracelet. In other embodiments, one or more detection units 32 or other sensors may be placed or built into a bed, a chair, an infant car seat, or other suitable clothing, furniture, equipment and accessories used by those susceptible to seizures. The detection unit 32 may comprise a simple sensor, such as an electrode, that may send signals to the base station 34 for processing and analysis, or may comprise a “smart” sensor having some data processing and storage capability. A detection unit 32 may include one or more smart client applications. In some embodiments, a simple sensor may be connected via wire or wirelessly to a battery-operated transceiver mounted on a belt or other garment or accessory worn by the person.

The system 30 may, for example, monitor the patient 48 while he or she is resting, such as during the evening and nighttime hours. If the detection unit 32 on the patient 48 detects a seizure, the detection unit 32 may communicate via wire or wirelessly, e.g., via a communications network or wireless link, with the base station 34, to a remote cell phone or other hand-held or desktop device via Bluetooth or other signal or simultaneously to a base station 34 and remote cell phone or other device. In some embodiments, a detection unit 32 may send some signals to the base station 34 for further analysis or for classification. For example, the detection unit 32 may process and use EMG signals (and additionally or in some embodiments, ECG, temperature, orientation sensors, saturated oxygen, and/or audio sensor signals) to make an initial assessment regarding the likelihood of occurrence of a seizure and may send those signals and its assessment to the base station 34 for separate processing, confirmation, or classification. However, in some embodiments, the detection unit 32 may be specifically designed to be capable of executing one or more seizure-detection routine to detect and/or detect and classify seizures using EMG and without relying on a plurality of other types of sensor data. Accordingly, battery resources may be conserved as compared to some other systems that may rely on multiple sensor and data types for detection and/or classification of seizure activity. In some embodiments, if the base station 34 confirms that a seizure is likely occurring, then the base station 34 may initiate an alarm for transmission over the network 50 to alert a designated individual by way of email, text, phone call, or any suitable wired or wireless messaging indicator. In some embodiments, an alarm may include a message that may indicate, by way of example, how an event was classified, one or more selectable states selected by a patient prior to a seizure event detection, a protocol for initiating a response, and combinations thereof

It should be appreciated that the detection unit 32 may, in some embodiments, be smaller and more compact than the base station 34, and it may be convenient to use a power supply with only limited strength or capacity. Therefore, it may be advantageous, in some embodiments, to control the amount of data that is transferred between the detection unit 32 and the base station 34 as this may increase the lifetime of any power supply elements integrated in or associated with the detection unit 32. In some embodiments, if one or more of the detection unit 32, the base station 34, or a caregiver, e.g., a remotely located caregiver monitoring signals provided from the base station 34, determines that a seizure may be occurring, video camera 38 may be triggered to collect video information of the patient. Or, other sensor systems, including, for example, systems which may be remotely located or disposed on or in the patient's body, may be activated.

The base station 34, which may be powered by a typical household power supply and may contain a battery for backup, may have more processing, transmission, and analysis power available for its operation than the detection unit 32 and may be able to store a greater quantity of signal history and evaluate a received signal against that greater amount of data. The base station 34 may communicate with an alert transceiver 40 located remotely from the base station 34, such as in the bedroom of a family member, or to a wireless device 42, 44 carried by a caregiver or located at a work office or clinic. The base station 34 and/or transceiver 40 may send alerts or messages to designated people via any suitable means, such as through a network 50 to a cell phone 42, PDA 44 or other client device. The system 30 may thus provide an accurate log of seizures, which may allow a patient's physician to understand more quickly the success or failure of a treatment regimen. Of course, the base station 34 may simply comprise a computer having installed a program capable of receiving, processing and analyzing signals as described herein and capable of transmitting an alert. A base station 34 may include one or more smart client applications. In other embodiments, the system 30 may simply comprise, for example, EMG electrodes as part of a device configured to transmit signals to a smartphone, such as an iPhone, configured to receive EMG signals from the electrodes for processing the EMG signals as described herein using an installed program application. In further embodiments, so-called “cloud” computing and storage may be used via network 50 for storing and processing the EMG signals and related data. In yet other embodiments, one or more EMG electrodes may be packaged together as a single unit with a processor capable of processing EMG signals as disclosed herein and sending an alert over a network. In other words, the apparatus may comprise a single item of manufacture that may be placed on a patient and that does not require a base station or separate transceiver. Or the base station may be a smartphone or tablet computer, for example.

In the embodiment of FIG. 2, collected signal data may be sent to a remote database 46 for storage. In some embodiments, signal data may be sent from a plurality of patients with epilepsy to a central database 46 and “anonymized” to provide a basis for establishing and refining generalized “baseline” sensitivity levels and signal characteristics of an epileptic seizure. The database 46 and base station 34 may be remotely accessed via network 50 by one or more remote computers 52 to allow updating of detector unit 32 and/or base station 34 software and data transmission. And, in some embodiments, the remote computer 52 or another computer may also serve to monitor exchange of data including alarm signals and EMG signal data between different devices associated with any number of designated individuals set to receive the signals. The base station 34 may generate an audible alarm, as may a remote transceiver 40 or detection unit 32. In some embodiments, wireless links may be two-way for software and data transmission and message delivery confirmation. Base station 34 may also employ one or all of the messaging methods listed above for seizure notification. The base station 34 or detection unit 32 may provide an “alert cancel” button to terminate an incident warning.

In some embodiments, a transceiver may additionally be mounted within a unit of furniture or some other structure, e.g., an environmental unit or object. If a detection unit 32 is sufficiently close to that transceiver, such a transceiver may be capable of sending data to a base station 34. Thus, the base station 34 may be aware that information is being received from that transceiver, and therefore base station 34 may identify the associated environmental unit. In some embodiments, a base station 34 may select a specific template file, e.g., such as including threshold values and other data as described further herein, that is dependent upon whether or not it is receiving a signal from a certain transceiver. Thus, for example, if the base station 34 receives information from a detector and from a transceiver that is associated with a bed or crib, it may treat the data differently than if the data is received from a transceiver associated with another environmental unit, such as, for example, clothing typically worn while an individual may be exercising or an item close to a user's sink where for example a patient may brush his or her teeth. A base station 34 may also send information to a detection unit 32 instructing the detection unit 32 to use one or more specific template files. More generally, a monitoring system may, in some embodiments, be configured with one or more elements with global positioning system (GPS) capability, and location or position information may be used to adjust one or more routines that may be used in a detection algorithm. For example, GPS capability may be included along with or among one or more microelectromechanical sensor elements included in a detection unit 32.

The embodiment of FIG. 2 may be configured to be minimally intrusive to use while sleeping or minimally interfere in daily activities, may require a minimum of electrodes such as one or two, may require no electrodes to the head, may detect a seizure with motor manifestations, may alert one or more local and/or remote sites of the presence of a seizure, and may be inexpensive enough for home use.

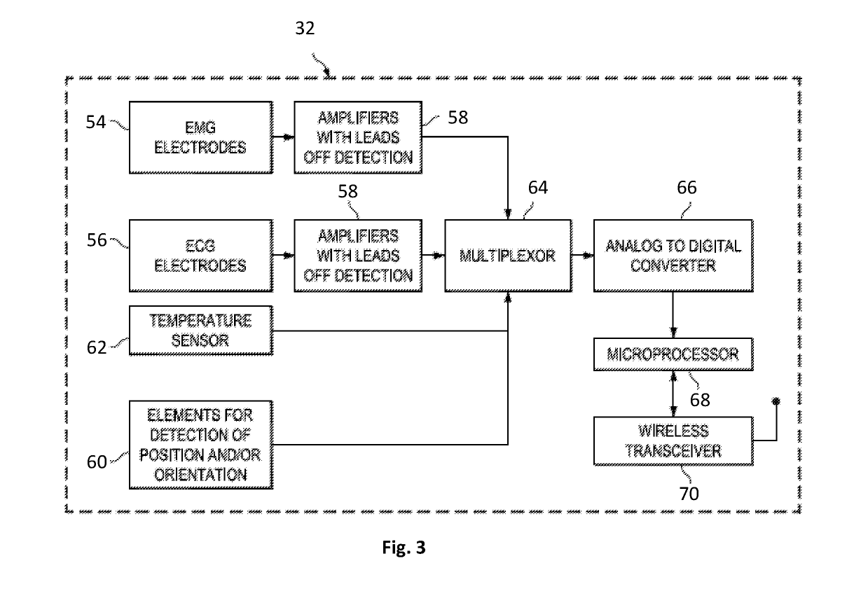

FIG. 3 illustrates an embodiment of a detection unit 32 or detector. The detection unit 32 may include EMG electrodes 54 and may also include, in some embodiments, ECG electrodes 56. The detection unit 32 may further include amplifiers with leads-off detectors 58. In some embodiments, one or more leads-off detectors may provide signals that indicate whether the electrodes are in physical contact with the person's body or otherwise too far from the person's body to detect muscle activity, temperature, brain activity, or other patient phenomena. In some embodiments, data derived from the leads-off detection may further be used to assist in classification of detected seizure-related events. The detection unit 32 may further include one or more elements 60, such as solid state microelectromechanical (MEMS) structures, configured for detection of position and/or orientation of the detection unit 32. For example, an element 60 may include one or more micromachined inertial sensors such as one or more gyroscopes, accelerometers, magnetometers, or combinations thereof.

The detection unit 32 may further include a temperature sensor 62 to sense the person's temperature. Other sensors (not shown) may be included in the detection unit 32, as well, such as accelerometers, microphones, and oximeters. Signals from EMG electrodes 54, ECG electrodes 56, temperature sensor 62, orientation and/or position sensors 60 and other sensors may be provided to a multiplexor 64. The multiplexor 64 may be part of the detection unit 32 or may be part of the base station 34 if, for example, the detection unit 32 is not a smart sensor. The signals may then be communicated from the multiplexor 64 to one or more analog-to-digital (A-D) converters 66. Analog-to-digital converters 66 may be part of the detection unit 32 or may be part of the base station 34. The signals may then be communicated to one or more microprocessors 68 for processing and analysis as disclosed herein. The microprocessors 68 may be part of the detection unit 32 or may be part of the base station 34. The detection unit 32 and/or base station 34 may further include memory of suitable capacity. The microprocessor 68 may communicate signal data and other information using a transceiver 70. Communication by and among the components of the detection unit 32 and/or base station 34 may be via wired or wireless communication.

In some embodiments, the exemplary detection unit of FIG. 3 may be differently configured. Many of the components of the detector of FIG. 3 may be in base station 34 rather than in the detection unit 32. For example, detection unit 32 may simply comprise an EMG electrode 54 in wireless communication with a base station 34. In such an embodiment, A-D conversion and signal processing may occur at the base station 34. If, for example, an ECG electrode 56 is included, then multiplexing may also occur at the base station 34.

In another example, the detection unit 32 of FIG. 3 may comprise an electrode portion having one or more of the EMG electrode 54, ECG electrode 56 and temperature sensor 62 in wired or wireless communication with a small belt-worn transceiver portion. The transceiver portion may include a multiplexor 64, an A-D converter 66, microprocessor 68, transceiver 70 and other components, such as memory and I/O devices (e.g., alarm cancel buttons and visual display).

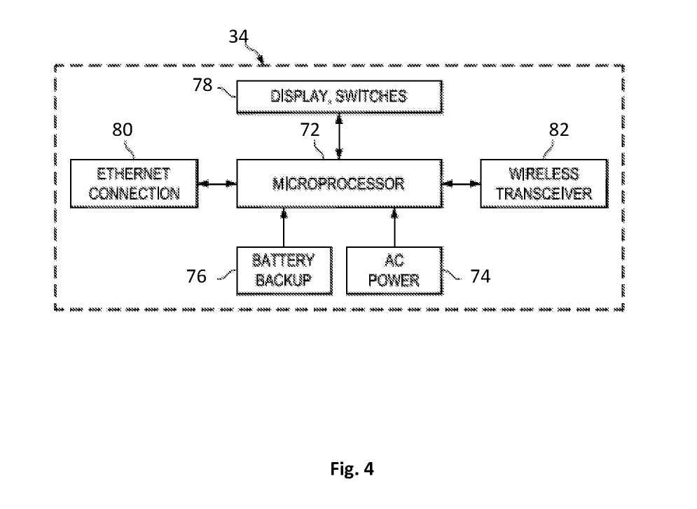

FIG. 4 illustrates an embodiment of a base station 34 that may include one or more microprocessors 72, a power source 74, a backup power source 76, one or more I/O devices 78, and various communications means, such as an Ethernet connection 80 and wireless transceiver 82. In some embodiments, the base station 34 may have more processing and storage capability than the detection unit 32 and may include a larger electronic display for displaying EMG signal graphs for a caregiver to review EMG signals in real-time as they are received from the detection unit 32 or historical EMG signals from memory. The base station 34 may process EMG signals and other data received from the detection unit 32. If the base station 34 determines that a seizure is likely occurring, it may send an alert to a caregiver via transceiver 82.

Various devices in the apparatus of FIGS. 2-4 may communicate with each other via wired or wireless communication. The system 30 may comprise a client-server or other architecture and may allow communication via network 50. Of course, the system 30 may comprise more than one server and/or client. In other embodiments, the system 30 may comprise other types of network architecture, such as a peer-to-peer architecture, or any combination or hybrid thereof.

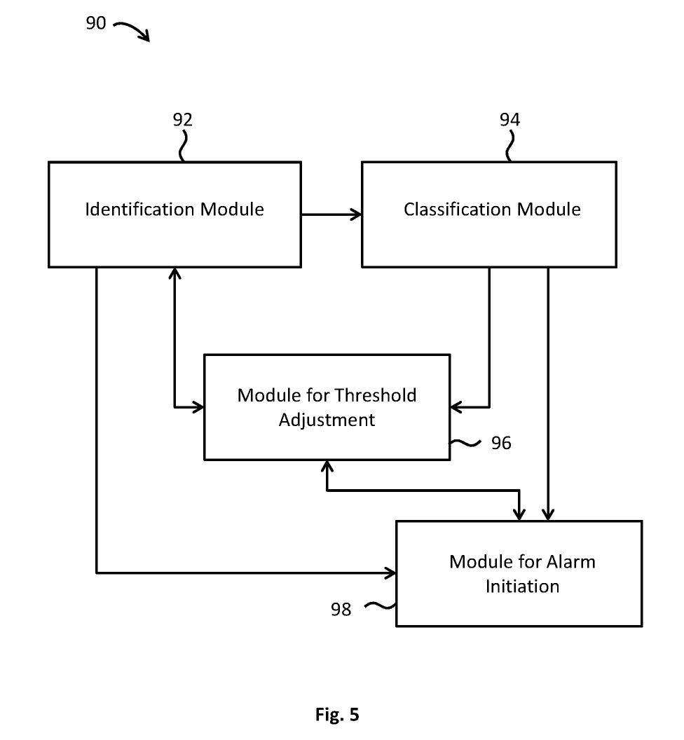

In some embodiments, one or more components in a seizure detection system may be organized into one or more system modules as shown in the system 90, which is described in relation to FIG. 5. For example, one or more of the apparatuses described above in relation to the system 30 may be included in the system 90. As shown therein, the system 90 may include each of an identification module 92 and a classification module 94. The modules 92, 94 may include one or more components, including, for example, one or more processors, transceivers, sensors, routers, other components, and combinations thereof, configured to execute one or more seizure-detection routines. For example, the modules 92, 94 may include one or more processors included in detection unit 32, base station 34, or a combination of both. Identification module 92 may be configured to detect seizure-related events and to initiate one or more responses based on their detection. Classification module 94 may be configured to classify detected seizure-related events as being associated with one or more types of physiological activity.

In some embodiments, responses initiated based on detection of seizure-related events using identification module 92 may include initiating execution of one or more seizure-detection routines configured for event classification and executable using classification module 94. In some embodiments, the one or more seizure-detection routines executable by the classification module 94 may, at least initially, oversee alarm or emergency responses. For example, classification of seizure-related events as being related to seizure-related physiological activity using classification module 94 may initiate the transmission of one or more alarms wherein a caregiver is instructed to check on the status of a patient.

In some embodiments, classification module 94 may also provide feedback to the identification module 92. For example, results of seizure-related event classification may be used to adjust detection conditions used by identification module 92. For example, in the system 90, adjustment of one or more detection conditions used in the identification module 92 may be controlled or supervised using a threshold adjustment module 96. The threshold adjustment module 96 may be configured to receive information regarding how detected seizure-related events were classified in the classification module 94. For example, a false positive detection may be logged when a seizure-event was identified using one or more seizure-detection routine in the module 92, but the classification module 94 determines that the event should be classified as a non-seizure movement. In some embodiments, threshold adjustment module 96 may respond to such an event or some number of events by automatically adjusting one or more thresholds. In some embodiments, threshold adjustment module 96 may evaluate or score how well identification module 92 functions in identifying seizure-related muscle activity that is truly representative of actual seizures or other activity when using one or more detection conditions (e.g., when using one or more seizure-detection routines and one or more thresholds or groups of thresholds). In some embodiments, threshold adjustment module 96 may generate or assemble detection condition data, including, for example detection conditions not previously executed in monitoring of a patient. In some embodiments, evaluation or scoring may be executed regularly, such as in regular intervals. In some embodiments, evaluation or scoring may be executed when triggered by some event, such as a false positive detection or reporting of one or more missed seizure, such as may be input by a caregiver or other persons.

By way of example, it may be, that, when using a seizure-detection routine and one or more thresholds, identification module 92 identifies seizure-related muscle activity with high sensitivity. However, based on classification information received from the classification module 94, threshold adjustment module 96 may determine that the identification module 92 only shows poor selectivity for identifying seizure-related events that are true seizures while discriminating EMG signals from non-seizure events. Accordingly, as further discussed below, classification module 94 may operate with a higher than desired duty cycle. And, in some embodiments, threshold adjustment module 96 may accordingly control or adjust one or more threshold settings associated with identification module 92, such as to reduce the duty cycle of operation of classification module 94 and/or to reduce data communicated between the modules 92, 94. In some embodiments, threshold adjustment module 96 may control or adjust one or more threshold settings in order to achieve one or more performance metrics of one or more seizure-detection routines when using one or more thresholds or groups of thresholds. The system 90 may be considered calibrated when the system 90 achieves the one or more performance metrics. In some embodiments, once the system 90 is calibrated, the system 90 system may operate without further execution of classification module 94. For example, once calibrated, system 90 may operate without execution of seizure-detection routines in the classification module 94. In other embodiments, the calibrated system 90 may continue to operate with execution of seizure-detection routines in the classification module 94. However, the system 90 may be calibrated so that those routines normally execute with a duty cycle of operation that is within one or more ranges or below a maximum duty cycle threshold. Thus, the system 90 may be enabled to execute a plurality of seizure-detection routines, including some that may be computationally intensive, but the system may be maintained within one or more power consumption or battery lifetime specifications.

In some embodiments, execution of alarm protocols may be supervised or controlled by an alarm initiation module 98. And, in some embodiments, threshold adjustment module 96 may communicate, directly or indirectly, with alarm initiation module 98. For example, in some embodiments, if a detection condition is found to perform with high sensitivity and high selectivity for epileptic seizures, alarm initiation module 98 may determine that identification module 92 may dictate transmission of alarms without significant risk of initiation of a false positive alarm. In some embodiments, a decision on whether identification module 92, classification module 94, or both may dictate one or more alarm protocols may be based on one or more performance metrics when using one or more detection conditions.

Threshold adjustment module 96 and alarm initiation module 98 may include one or more processors, including, for example, one or more processors included in detection unit 32, base station 34, or a combination of both. For example, in some embodiments, threshold adjustment calculations may be executed using a processor included in base station 34. Accordingly, base station 34 may receive classification data for seizure-related events. Thus, at least in some embodiments, selectivity for detection of seizure-related events that are most important (e.g., those truly related to a seizure) may affect an amount of data transmitted to the base station 34.

In some embodiments, identification module 92 may include one or more processors included in one or more detection units 32. Some seizure-detection routines executable in identification module 92 may compare one or more property values determined from an EMG signal to one or more thresholds. In some embodiments, a property value determined in one or more of the seizure-detection routines executable in identification module 92 may be an amplitude of an EMG signal, a number of zero crossings exhibiting a hysteresis, a T-squared statistical value determined from an EMG signal, or a principal component value determined from an EMG signal. In some embodiments, amplitudes or other statistical values of an EMG signal may be baseline corrected or scaled in some way. For example, an amplitude may be scaled in terms of the uncertainty or noise of a baseline or reference region as may be used to calculate a signal-to-noise ratio or other scaled factor.

In some embodiments, seizure-detection routines executable in identification module 92 may be characterized by a detection interval. For example, as also described in FIG. 1A, following collection of data in a given detection interval, a processor including instructions to execute a seizure-detection routine may evaluate whether EMG data collected in a detection interval indicates the presence of one or more seizure-related events. Thus, a detection interval may refer to a time interval that corresponds to an amount or maximum amount of data used in any individual calculation of whether seizure-related muscle activity and associated seizure-related events may be detected. In some embodiments, seizure-detection routines executable in identification module 92 may desirably respond with minimal lag between physical manifestation of physiological activity and its detection. The seizure-detection routines executable in identification module 92 may also be configured to run continuously or nearly continuously without using prohibitive amounts of energy from a battery or other energy resource. Accordingly, in some embodiments herein, a seizure-detection routine executable in identification module 92 may only process limited amounts of data in detection of seizure-related muscle activity. For example, in some embodiments, a seizure-detection routine executable in identification module 92 may operate using detection intervals including less than about 10 seconds, less than about 5 seconds, less than about 3 seconds or less than about 1 second worth of data from a collected EMG signal.

Classification module 94 may include one or more processors configured to execute one or more other seizure-detection routines. In some embodiments, the seizure-detection routines executable in classification module 94 may operate selectively when gated or initiated by detection of seizure-related muscle activity, such as may be detected using identification module 92. Accordingly, in some embodiments of operation of the system 90, a duty cycle of operation, or ratio of times in which collected data was processed by a module to a total collection time of all data in a given period, for classification module 94 may be relatively low. For example, if only 2 minutes of collected data is processed in a module over a 200-minute monitoring period, the module may be characterized as having a duty cycle of operation of 2:200 or 1:100. In some embodiments, classification module 94 may, when calibrated, generally operate with a duty cycle of operation of less than about 1:10 or less than about 1:100. And, in some embodiments, a system 90 may automatically calibrate based on one or more performance metrics, the system deeming calibration to be completed when the one or more performance metrics are within one or more performance metric thresholds and while maintaining a duty cycle of operation for one or more seizure detection routine used in classification module 94 of less than about 1:25, less than about 1:50, or less than about 1:100.

In some embodiments, classification module 94 may include one or more processors that may also be included in identification module 92. For example, in some embodiments, both classification module 94 and identification module 92 may include one or more processors associated with one or more common detection units 32. In other embodiments, one or more processors may be included in the classification module 94 that are not included in identification module 92. For example, in some embodiments, identification module 92 may include processing of signals using detection unit 32, and at least some seizure-detection routines executable in classification module 94 may be executed at a base station 34 processor and/or other processor different than one included in a patient mounted or disposed detection unit 32. Thus, at least in some embodiments, selectivity for detection of seizure-related events of a given type may affect how often seizure-related event data may be transmitted, such as to a base station 34 or other device. In some embodiments, one or more seizure-detection routines associated with classification module 94 may be executed using one or more processors configured to receive seizure-related event data directly or indirectly from a transceiver. For example, one or more processors included in classification module 94 may be part of a base station 34, remote computer 52, part of a belt-worn or other device that may be worn or held by a patient, or combinations thereof. Thus, processors in classification module 94 may operate on an EMG signal directly collected by an EMG electrode, operate on an EMG signal transmitted to a processor that is physically separated from an EMG electrode, or operate on a combination of both signal types.

In some embodiments, classification module 94 may be configured to execute one or more seizure-detection routines that may include detection and qualification of signals that may be associated with a particular part of a seizure such as clonic-phase activity, a patient's progression during seizure recovery and/or one or more post-seizure activities. For example, levels of clonic-phase activity may be determined. In some embodiments, signals that may be associated with a particular part of a seizure such as clonic-phase activity may be identified by detecting samples of signal that may include a leading edge of a peak, a trailing edge of a peak, and/or both leading and trailing edges of a peak. Samples of signal may further be qualified as being associated with the clonic-phase of a seizure, as being associated with one or more parts of the clonic-phase of a seizure, or as being associated with activity commonly mistaken for the clonic-phase of a seizure, such as a PNES event. Qualification of data may include a comparison of one or more properties or criteria of individual samples of signal to one or more qualification thresholds. For example, in some embodiments, samples of signals may be qualified based on whether samples of an EMG signal meet criteria suitable to be qualified as being related to the clonic-phase of a seizure or part similar to the clonic phase of a seizure such as may be present during PNES. For example, samples of an EMG signal may be qualified based on a comparison of criteria values to thresholds; wherein the criteria values include a duration width and one or more of a signal-to-noise ratio and an amplitude; and wherein the thresholds include a minimum duration width, a maximum duration width, and one or more of a minimum signal-to-noise ratio, minimum amplitude, and maximum amplitude.

In some embodiments, qualification of data may also include grouping of more than one sample together, with qualification accomplished by comparing an aggregate property of a group of samples to an aggregate property threshold. For example, included among aggregate qualification threshold values that may be used to qualify samples in a group are one or more of a minimum deviation value calculated from duration widths of samples or parts of samples, a maximum deviation value calculated from duration widths of samples or parts of samples, a minimum rate of sample repetition, a maximum rate of sample repetition, a minimum regularity of one or more sample characteristics, a maximum regularity of one or more sample characteristics, and/or combinations of the aggregate qualification threshold values thereof.

Further, in some embodiments, qualification of data may include a combination of individual sample qualification and qualification of sample groups. In some embodiments, once qualified as being associated with the clonic-phase of a seizure or one or more parts of the clonic-phase of a seizure, a qualified-clonic-phase burst count or one or more other metrics of burst activity may be determined. For example, a qualified-clonic-phase burst activity level may be compared to a threshold activity level in order to identify or classify data as associated with a physiological type that includes a clonic phase portion of a seizure, such as a GTC seizure.

In some embodiments, qualification of data may be performed in discrete steps. For example, samples may be organized so that at least some samples may be removed as part of a qualification or prequalification step. Organization of samples may include removal of one or more samples from a larger group of samples as may be used to construct test groups including various numbers of samples of signal. For example, a set of data may include a full set of detected samples of signal or a set with some samples removed in order to construct test groups of data. Constructed test groups may then be compared to patterns of samples associated with seizure-related muscle activity related to different physiological conditions.

In some embodiments, samples may be excluded from one or more groups based on certain criteria. For example, a procedure may order samples based on one or more peak characteristics. Based on that ordering, a list of candidate samples including peaks most likely to be spurious (e.g., not related to physiological activity) may be generated. For example, a group of samples may be ordered based on peak or elevated sample portion duration width, and that ordering may be used to identify that the duration width of a majority of samples are clustered around a central range, but one or more samples may be characterized as having an elevated portion with a duration width outside of that central range. Samples outside of the most common range may be removed or first removed in generation of test groupings. For example, in some embodiments, one or more samples may be excluded from some groups as part of routines configured, for example, to search for particular patterns of activity. Removal of prequalified samples and test group construction, including as related to identification of patterns in data that may be noisy or intermittent, is also described in Applicant's U.S. patent application Ser. No. 14/920,665, filed on Oct. 22, 2015.

In some embodiments, classification module 94 may include one or more processors configured to execute one or more of the seizure-detection routines as also described in Applicants' International Application PCT/US17/28429, filed on Apr. 19,2017. As also described therein, in some embodiments, seizure-detection routines may include wavelet transformation of EMG signals, organization of transformed signals into a high frequency group of data and one or more lower frequency groups of data, determining the strengths or magnitudes of signals in one or more of the organized groups, scaling the strengths or magnitudes of the signals, and comparing magnitudes and/or scaled magnitudes of the signals to one or more thresholds.

For example, in some embodiments, EMG signal data may be processed with a Morlet wavelet, which may be used to express the complex power in frequency over time of the EMG signal data. The transformed data may then be organized into several different groups of data. For example, in some embodiments, a high frequency group of data may include signal components in a range from about 150 Hz to about 260 Hz. A lower frequency group may include signal components in a range of frequencies from about 6 Hz to about 70 Hz.

In some embodiments, the transformed signal may be integrated over boundaries with respect to frequency and/or time. For example, the transformed signal may be integrated over some increment or unit of time (e.g., an increment or unit of time within an overall analysis window) and also over one or more of the aforementioned frequency ranges or other suitable frequency ranges. The aforementioned integrations may be repeated for other time increments or time units within an overall analysis time window. Thus, an integrated magnitude or strength of signal in one or more bands may be tracked over any part of an analysis time window. In some embodiments, the integrated magnitude or strength of signal in the one or more bands may then be scaled. Scaled magnitudes may be used (or used together with non-scaled magnitude data) to determine characteristics of seizure-related events and to classify seizure-related events as associated with one or more physiological activity types. For example, scaling of magnitude data for one or more frequency components may include dividing magnitude data by a maximum magnitude value achieved over time within an analysis time window or a maximum magnitude value achieved over time within a part of an analysis window.

Magnitudes and/or scaled magnitudes may be compared to thresholds in order to determine whether a tonic phase or clonic phase may be present. For example, in some embodiments, if a scaled magnitude of greater than about 0.8 is determined for a high frequency component of an EMG signal, a tonic phase may be classified. In some embodiments, if a scaled strength magnitude of greater than about 0.8 is determined for a lower frequency component of an EMG signal, a clonic phase may be classified. Other rules may be established for characterization of seizures where more than one phase of a seizure is determined to be concomitantly active. For example, in some embodiments, if both tonic and clonic phases are determined to be active using the above rules, the phase may be described as tonic, unless the scaled strength of low frequency components is found to be greater than about 1.25 times higher than the scaled strength of the high frequency components. Accordingly, phases of a GTC seizure may be automatically characterized throughout the course of the seizure. And, notably, the duration of the seizure and different phases of a seizure may be determined. A detected seizure-related event may then, for example, be classified as a GTC seizure if each of a clonic phase and tonic phase is detected, and the duration of a GTC seizure and parts thereof may be reliably determined.

In some embodiments, one or more indices of activity for the tonic phase and/or clonic phase activity of a seizure may be calculated. For example, indices of seizure activity for the tonic and clonic phases of a seizure may be calculated as shown in Equation 1 and in Equation 2:

I_{T}=k1∫(Scaled Magnitude (High Freq.) dt where, 0<t<xx min Eqn. 1

I_{c}=k2∫(Scaled Magnitude (Low Freq.) dt where, 0<t<xx min Eqn. 2

In Equation 1, the tonic phase index (IT) includes a scaling factor k1 and an integrated value across time for the scaled signal magnitude calculated for a high frequency component of wavelet-transformed EMG signal data. In some embodiments, magnitude data may be substituted for scaled magnitude data in Equation 1 to derive a tonic index. In Equation 2, the clonic phase index (Ic) includes a scaling factor k2 and an integrated value across time for the scaled signal magnitude calculated for a lower frequency component of wavelet-transformed EMG signal data. In some embodiments, magnitude data may be substituted for scaled magnitude data in Equation 2 to derive a clonic index.

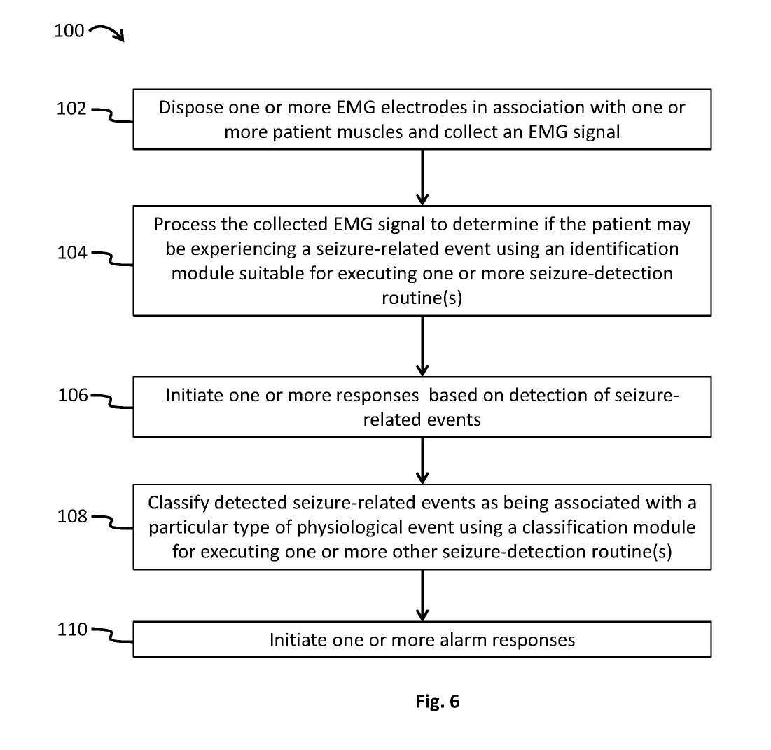

In some embodiments, system 90 or another suitable system may be used in execution of a method 100 for detection of seizure-related muscle activity as shown in FIG. 6. For example, as shown in step 102, the method 100 may include disposing one or more EMG electrodes in association with one or more patient muscles and collecting of an EMG signal. The electrodes may be suitably configured to transduce energy associated with muscle activation into a form that may be electronically processed. For example, in some embodiments, bipolar differential electrodes may be disposed on the skin of a patient near a patient's biceps, triceps, other patient muscle that may be activated during a seizure, and/or any combination of the muscles thereof. For example, in some embodiments, EMG electrodes may be disposed on muscles on opposite sides of the body or on one or more pairs or groups of muscles for which information is desired about how coordinated or synchronized muscle activity may be.

In some embodiments, collected EMG signal may be processed in step 102 to produce a digital EMG signal suitable for digital processing. For example, in some embodiments, a collected EMG signal may be amplified and processed using an analog-to-digital converter. In some embodiments, operations such as rectification, low pass filtering, or other operations that may be used to shape or condition an EMG signal in some other suitable way within the ordinary level of one skilled in the art of electronic signal processing may also be performed in the step 102.

In step 104, collected EMG signal may be processed to determine if a patient may be experiencing a seizure-related event. For example, in some embodiments, any of the various suitable seizure-detection routines described as executable using identification module 92 may be executed in step 104. For example, seizure-detection routines executed in the step 104 may evaluate one or more segments of EMG signal data in order to determine an amplitude property value of the EMG signal or some statistical values calculated therefrom, such as a T-squared statistical property value or principal component property value. Or, a property value for a number of zero crossings exhibiting a hysteresis may be determined. Property values may be compared to one or more thresholds in order to determine if a seizure-related event is detected.

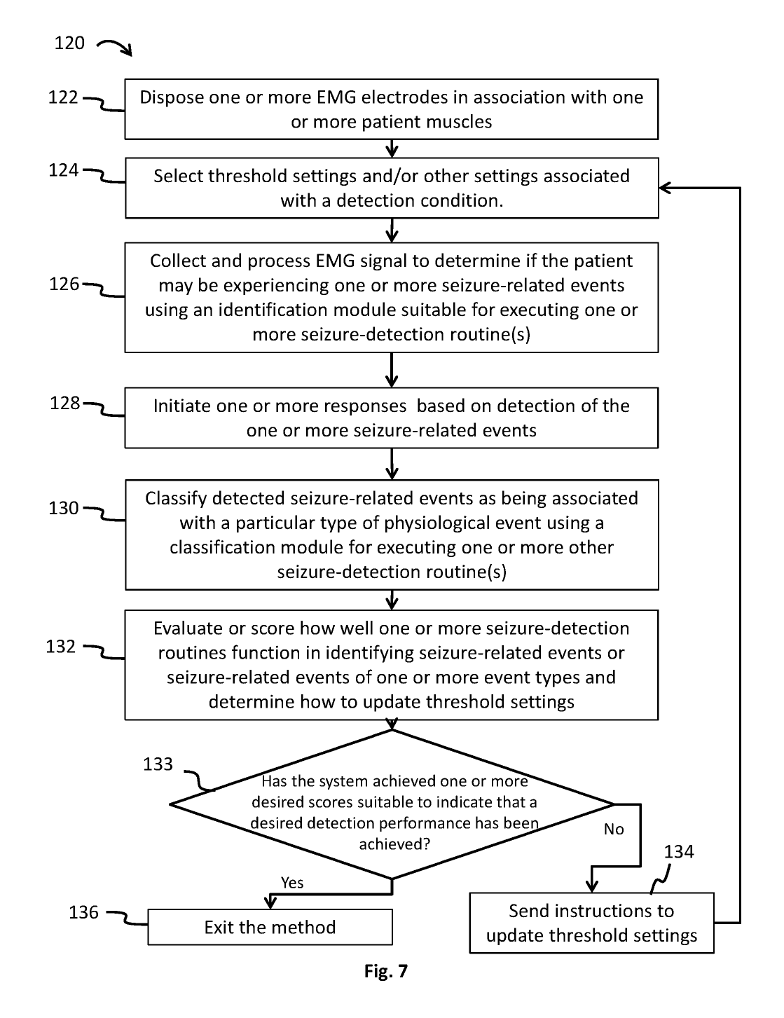

In some embodiments, the one or more thresholds may be part of a threshold settings file. A threshold settings file may include instructions for applying one or more thresholds or groups of thresholds in one or more seizure-detection routines. Threshold settings used in the one or more seizure-detection routines in step 104 may be fixed or adjustable. Or, some seizure-detection routines may use fixed thresholds whereas other routines may use adjustable threshold settings. In some embodiments, the thresholds settings may be controlled or set using threshold adjustment module 96. For example, threshold adjustment module 96 may periodically update one or more threshold settings based, for example, on one or more performance metrics of one or more seizure-detection routines when using one or more thresholds or groups of thresholds. For example, a sensitivity or rate of detection of a seizure-detection routine when using a certain threshold may be evaluated with respect to one or more types of seizure-related events as classified using classification module 94. Some embodiments of methods using classification module 94 are also described herein in greater detail in reference to method 120 as shown in FIG. 7, such as in classification step 130 therein. Performance metrics, useful in some embodiments wherein threshold settings are adjusted, are also described herein in greater detail in reference to method 120, such as in step 132 therein.

In step 106, one or more responses may be initiated based on whether one or more seizure-related events were detected. In some preferred embodiments, a response to detection of one or more seizure-related events may include the initiation or triggering of execution of one or more additional seizure-detection routines, such as described in relation to step 108. However, in some embodiments, other responses may additionally or alternatively be executed. For example, in some embodiments, a response initiated in step 106 may include one or more of initiation of one or more emergency or warning alarms, execution of one or more strategies to treat or cause cessation of seizure activity, verification or detection of a patient's position within a monitoring location or other locale, verification or detection of a patient's orientation, adjustment of a rate at which a patient's position is determined, collection of data or adjustment of a setting associated with one or more physiological sensors, collection of video and/or audio data, testing of battery or power resources associated with detection unit 32, testing of communication strength or fidelity between detection unit 32 and one or more base stations 34 or routers, transmission of raw or processed EMG data to one or more remote caregiver devices (e.g., devices 42, 44), determining of a location of a patient with respect to one or more environmental transceivers, verification or detection of the position of one or more caregivers, execution of one or more routines configured to examine a patient for non-seizure sources or noise in an EMG signal, testing of one or more electrodes for contact integrity with a patient's skin, verification of whether a patient has selected one or more selectable settings, other suitable responses, and any combinations thereof.

In some embodiments, initiation of a warning or emergency alarm may be included among responses initiated in the step 106. Initiation of a warning or emergency alarm may be controlled using any of various alarm protocols. Execution or selection of alarm protocols may, for example, be controlled or supervised using a suitable processor, such as, for example, a processor included in module for alarm initiation 98. In this disclosure, an emergency protocol includes any transmission that instructs or is predetermined to instruct a caregiver to physically check on the health of the patient unless that action is actively canceled by a person. In some embodiments, an emergency protocol may include scheduling transmission of an emergency message, but one or more caregivers may be provided information to actively terminate the message. For example, a remote caregiver who has been provided with classified EMG data or other data may instruct other caregivers, such as first responders, that an emergency response is not warranted or terminate a message otherwise queued for transmission to other caregivers. Some embodiments herein are particularly configured to facilitate such protocols, because, for example, an emergency message may be queued for transmission, such as queued for transmission after some set delay, but during that period classification routines may complete execution and classification data may be provided to a caregiver or used to automatically cancel a queued message. For example, at some time between queuing of an emergency alarm message and transmission of that message, a remote caregiver may be provided information that the event was classified as a low-risk or non-seizure event. In contrast to an emergency message, messages that may inform a caregiver that a patient may be experiencing abnormal motor manifestations or that a device may be detecting abnormal activity, but that do not instruct the caregiver to physically check on the health of the patient, may be part of a warning protocol. Of course, one or more caregivers may choose to check on a patient or instruct another caregiver to physically check on a patient in response to a warning message. However, some of the methods herein, including methods that facilitate both detection and classification of seizure-related events using a mobile detection device may be ideally configured to provide messages that instruct a caregiver with an expected protocol of response. By way of example only, some warning messages may be provided in response to a detection of a seizure-related event where the system has been logged in a particular patient state, such as a state where the patient is at home in the presence of another person or sleeping. However, a response may be updated if upon classification of a detected seizure-related event, a GTC seizure is confirmed or where a seizure exhibits one or more characteristics that may increase a risk of adverse effects of a seizure, such as a particularly intense clonic phase or tonic phase that is particularly extended in duration.

In some embodiments, a message instructing that an emergency response is initiated may be queued for transmission in the step 106 or a warning period may be established as a response in step 106. For example, an emergency message may be queued for transmission after some suitable period of time, such as a period of time of about 30 seconds to about 5 minutes. As noted above, a person, such as a local or remote caregiver, may actively cancel an alarm at some time between the time an emergency message is queued and when the emergency alarm message is scheduled for sending. Alternatively, a warning period may be established wherein an alarm may be sent at the completion of the warning period, but the event triggering initiation of the warning period may later be classified as a non-seizure event or other event that may not demand an alarm, and the alarm may be automatically canceled. In contrast to a queued emergency response, a possible alarm issuing from a warning period may be automatically canceled without direct intervention by a person.

In the step 108, one or more additional seizure-detection routines may be executed. For example, in some embodiments, any of the various suitable seizure-detection routines described as executable using classification module 94 may be executed in step 108. For example, seizure-detection routines executed in the step 108 may include one or more seizure-detection routines wherein wavelet analysis is performed on EMG signal data, strengths or magnitudes of signals in one or more high frequency and/or one or more lower frequency bands are determined, and trends in one or more clonic and/or tonic indices are determined. By way of further example, one or more levels of qualified clonic-phase bursts may be determined based on qualification of samples of EMG signal, which may be done using individual or aggregate properties of EMG signal samples. As further described herein, qualification techniques may or may not involve more computationally demanding operations such as construction of multiple test groups and pattern recognition.

In the step 110, one or more alarm responses or protocols may be initiated. Execution or selection of alarm protocols may, for example, be controlled or supervised using alarm initiation module 98. For example, in some embodiments, one or more seizure-detection routines executed in step 108 may indicate that a seizure-related event may properly be classified as a GTC seizure event or some other event demanding a response. Accordingly, an appropriate alarm response may be initiated in step 110. For example, in some embodiments, a response to classification of a seizure-related event as a GTC seizure may include instructing one or more caregivers to check on the status of a patient. In some embodiments, an alarm response may include sending raw or processed EMG data to a remote caregiver. For example, in some embodiments, one or more of the devices 32, 34, and 40 may, directly or indirectly, transmit raw or processed EMG data to one or more caregiver devices 42, 44. In some embodiments, once a seizure-related event is classified, including as an event type demanding a certain response, raw and/or processed EMG data may be transmitted to a remote computer 52 for immediate review. For example, EMG data may be transmitted to remote computer 52 and processed to verify the results of seizure-related event classification or processed to determine other information. Information may then be organized for transmission to one or more caregivers tasked with providing medical care to the patient. For example, an immediate emergency responder or medical doctor may timely receive information on how long the patient may have been experiencing a seizure or how long the seizure or individual phases of a seizure were active before the patient began receiving care. In some embodiments, data may be sent automatically to a caregiver and within some critical time period, such as within about 5 minutes or some other time period so that it may be used by caregivers tasked with immediate care of the patient. In some embodiments, where a detected seizure-related event is classified as being either of a non-seizure movement or a PNES event, a response may include logging the event as appropriately classified and/or storing associated data for further review. However, some of those events may terminate without an alarm instructing one or more caregivers to check on the status of a patient.

In some embodiments, a detected seizure-related event may be partially classified at a first time, but the partially-classified seizure-related event may be additionally classified using one or more routines executed at one or more later times. In some of those embodiments, additional classification of a detected seizure-related event may be executed automatically without the need for a human operator. Alternatively, classification data may be reviewed by someone trained to evaluate EMG data.

In some embodiments, partially-classified EMG data may be classified to the extent necessary to determine an immediate or suitably timed response to a detected seizure-related event. For example, in some embodiments, non-seizure movement activity and PNES activity may be grouped together during real-time analysis of seizure-related events because, at least for some patients or for patients in some selectable states, a common response may be initiated in response to either activity. For example, a response may include logging the events without instructing one or more caregivers to check on the status of a patient or canceling a queued alarm message if such an alarm were scheduled for transmission. However, it may still be useful to distinguish between those activity types by additionally classifying data at one or more later times.

In some embodiments in which a warning message has been sent prior to real-time classification of seizure-related events, a response initiated in step 110 may include automatically canceling transmission of an alarm message if classification results in a determination that the event is a non-seizure movement or other event that does not immediately threaten the health status of a patient. In some embodiments in which an emergency message has been queued for transmission, a response initiated in step 110 may include sending a caregiver a message communicating how the event was classified. For example, the remote caregiver may be a doctor or other person trained to evaluate a medical situation, such as a person trained to interpret EMG and/or other sensor data. In some embodiments, a remote caregiver may be automatically provided or given the option to request that collected EMG and/or other sensor data be transmitted to them. The remote caregiver may further be given an option to cancel a queued emergency alarm. For example, in some embodiments, a remote caregiver may cancel a queued emergency alarm by pressing only one or a limited number of alarm cancellation buttons or using some other simple and efficient signaling method. Notably, in some cases, cancellation of a queued alarm may involve revoking a plurality of messages prepared for transmission and designated for delivery to multiple persons, such as caregivers or other persons designated to receive messages. In some embodiments, systems described herein may be configured to log a list of queued or transmitted messages and associated caregivers and adjust the queued or transmitted messages, e.g., cancel messages or send appropriate additional messages, in batch form such as to one or more designated groups, based on further system or caregiver inputs.

Other embodiments of methods used in seizure detection are described in method 120 shown in FIG. 7. In some embodiments, method 120 may be executed using system 90 or another suitable system may be used. In some embodiments, method 120 may be used for adjusting one or more threshold settings used in seizure detection. For example, in some embodiments, method 120 may be executed during one or more training or reference periods wherein a detection system may execute a selection of initial threshold settings and/or adjustment of initial threshold settings used for patient monitoring. In some embodiments, method 120 may comprise a method for automatic calibration of threshold settings and for monitoring of a patient for detection of seizure activity, the monitoring of the patient uninterrupted during calibration.

In some embodiments, method 120 may be configured for execution when a patient engages in one or more activities, including, for example, starting a new treatment regimen, starting a new monitoring session, changing of one or more medications, and any combinations thereof. In some embodiments, method 120 may be configured for execution when one or more events occur, including, for example, when a patient or caregiver identifies that one or more false positive alarms have been initiated. In some embodiments, method 120 may be executed regularly as part of patient monitoring or as part of one or more regular or periodic calibration routines.

In some embodiments, method 120 and/or steps in the method 120 may be executed together with one or more steps executed in method 100. For example, as already described herein, in some embodiments of method 100, as shown in step 104, threshold settings may be adjustable. And, in some embodiments, the adjustable threshold settings therein may be determined as described in method 120. For example, updating or adjustment of thresholds (as described in step 134 of the method 120) may produce adjusted thresholds that may be applied in step 104 of method 100. In some embodiments, steps 102 through 108 and steps 122 through 130 or other steps of methods 100, 120 may involve at least some of the same or similar processing operations. For example, in some embodiments, detection of seizure-related events in steps 104, 126 and classification of seizure-related events in steps 108, 130 may involve some of the same or similar seizure-detection routines.

In step 122, EMG electrodes may be disposed in association with one or more patient muscles. The electrodes may be suitably configured to transduce energy associated with muscle activation into a form that may be electronically processed. For example, in some embodiments, bipolar differential electrodes may be disposed on the skin of a patient near a patient's biceps, triceps, other patient muscle that may be activated during a seizure, and/or any combination of the muscles thereof.

In step 124, one or more thresholds and/or other settings associated with a detection condition may be selected or applied for use. In some embodiments, the one or more thresholds may be designated in a threshold settings file. A threshold settings file may include instructions for applying one or more initial threshold settings and/or other threshold settings in a seizure-detection routine. The selection of threshold settings may, for example, be controlled using one or more appropriate processors, such as, for example, one or more processors that may be included in threshold adjustment module 96. An initial threshold setting may include one or more thresholds or groups of thresholds which may be applied for use in a seizure-detection routine executed during one or more calibration periods within a patient monitoring regimen. In some embodiments, initial threshold settings may be applied the first time a patient begins a treatment or monitoring regimen. However, in some embodiments, initial thresholds may be applied at other times in a patient's treatment regimen. For example, if a patient identifies the presence of an undesirable number of false positive detections in an already calibrated system, a detection system may automatically or manually be set to apply or reapply an initial thresholds settings file in step 124 and remaining steps in method 120 may be executed. In some embodiments, calibration may be executed without interrupting patient monitoring.

Advantageously, noting that some procedures described herein for setting initial settings may be inappropriate for some users, systems and methods herein may be configured to optimize a device based on any various procedures for setting initial threshold settings. In some embodiments, one or more initial threshold settings may be predetermined for use. For example, the one or more initial threshold settings may be predetermined based on empirically collected data. For example, an initial threshold setting may be obtained or selected based on historical data for a patient demographic or historical data for all patients. For example, a patient may be defined by various characteristics including, for example, any combination of age, gender, ethnicity, weight, level of body fat, fat content in the arms, fat content in the legs, or fitness level, or the patient may be defined by other characteristics. And, patients that share one or more of the above characteristics may be grouped together and historical data for the group evaluated in order to determine threshold settings. The patient's medical history, including, for example, history of having seizures, current medications, or other factors, may also be considered in selection of threshold settings. For example, all patients that share one or more common factors based on medical history may be grouped together, and historical data for the group may then be pooled and evaluated in order to determine one or more initial threshold settings.

In some embodiments, one or more initial threshold settings may be selected in step 124 based, at least in part, on one or more particular patient movements or operations executed during one or more training or reference periods. For example, a patient may execute one or more maximum voluntary contractions to establish, set, or adjust one or more initial threshold settings. MVC is related to the maximum force a patient may apply during a voluntary contraction. The strength of muscles may vary from individual to individual, and the amplitude of an EMG signal, or other statistical value calculated therefrom, may also vary. Measurement of electromyography data for a patient during maximum voluntary muscle exertion (and adjusting sensitivity settings accordingly) may customize the technique to an individual's musculature. For example, in some embodiments, an initial T-squared threshold value may be set based upon a T-squared statistical value calculated from EMG data obtained while an individual is at rest and while an individual is undergoing an MVC.

In some embodiments, selection of initial threshold settings in step 124 may include monitoring a patient in a controlled setting such as a hospital, and data, such as data derived from EMG electrode outputs, may be collected and correlated with the presence or absence of seizures. From that data, an operator or software may generate an initial threshold setting file or select an appropriate file from a list of pre-generated templates, including initial thresholds.

Once threshold settings are selected in step 124, the selected settings may be applied and used (as described in step 126) to determine if a patient may experience one or more seizure-related events. In some embodiments, some steps in method 120 may operate in an iterative manner. For example, step 124 may receive instructions to update or change one or more threshold settings. Accordingly, in some cases where method 120 operates in an iterative manner, threshold settings applied in an earlier iteration of step 124 may be referred to as initial thresholds, earlier used thresholds or first thresholds, and updated thresholds or second thresholds may be applied in one or more additional iterations of the method 120.

Once one or more thresholds are selected for use in step 124, EMG signal may be collected in step 126. Moreover, as also shown in step 126, EMG signal may be processed to determine if a patient may be experiencing one or more seizure-related events. For example, seizure-detection routines executed in the step 126 may be used to evaluate one or more property values of said EMG signal and compare the one or more property values to one or more thresholds for detection of one or more seizure-related events. In some embodiments, seizure-detection routines executed in the step 126 may be used to process EMG signals in order to determine one or more amplitude values or one or more statistical values calculated therefrom, such as a T-squared statistical value or principal component value. The aforementioned values or other suitable property values may be compared to the one or more of the thresholds (e.g., thresholds as selected previously in step 124) in order to determine if a seizure-related event is detected. In some embodiments, the one or more seizure-detection routines executed in step 126 may operate in a substantially continuous manner. For example, once executed, step 126 may operate substantially continuously whereas other steps may operate when initiated or triggered.

In a step 128, one or more responses may be initiated based on detection of one or more seizure-related events. For example, in some embodiments, a response to detection of one or more seizure-related events may include initiation of one or more seizure-detection routines suitable for classification of detected seizure-related events. In some of the embodiments herein, any of a number of suitable seizure-detection routines executable using classification module 94 may be initiated in the step 128. In some embodiments, a response initiated in step 128 may include transmission of a collected EMG signal to one or more processors, including, for example, one or more processors configured to execute one or more seizure-detection routines suitable for classification of detected seizure-related events. For example, EMG data may be transmitted to one or more processors associated with a different detection unit 32 than the detection unit 32 from which transmitted data was collected, transmitted to a processor associated with base station 34, transmitted to another processor operatively in communication with network 50, transmitted to one or more processors associated with a belt-worn processor unit, or transmitted to combinations of the processors thereof. In other embodiments, detection of seizure-related events (step 126) and classification of events (step 130) may be executed using a common detection unit 32. For example, each of detection of seizure-related events (step 126) and classification of events (step 130) may be executed using one or more microprocessor 68.

Some of the embodiments where transmission of an EMG signal is included among responses in step 128 may be particularly useful where inter-muscle coherence is used to classify detected seizure-related events (as described herein in relation to a step 130), or in other embodiments, where, for example, EMG data may be evaluated on more than one detection unit 32 on different muscles, and where EMG signals on the different muscles may be examined together to classify events in step 130. For example, in those embodiments, it may be particularly beneficial to accurately control and adjust threshold settings used in step 126 because transmission of data between detection units 32 may place an added burden on limited power resources.

In some embodiments, a response initiated in step 128 to detection of one or more seizure-related events may include communication with one or more processors configured to organize one or more alarm responses. For example, information concerning detection of one or more seizure-related events may be communicated to alarm initiation module 98. Thus, it should be appreciated that, in some embodiments, where method 120 may be executed to calibrate a device or set threshold settings, a patient may still be monitored for seizure activity. Accordingly, active alarms may be transmitted in order to provide medical care to a patient should the patient experience a seizure-related event.

In some embodiments, a response initiated in step 128 may be based on one or more estimates or measurements of remaining battery resources. For example, if a detection unit 32 has used more than some critical level of available battery resources, a response to detection of one or more seizure-related events may include initiation of one or more alarms. However, if less than some critical amount of available battery resources has been used, a response to detection of one or more seizure-related events may include additional processing, including, for example, classification of the one or more events as described in step 130. And, in some embodiments, an alarm may be issued only after the one or more seizure-related events have been classified. Accordingly, in some embodiments, a decision on whether detected seizure-related events, classified seizure-related events, or both may initiate an alarm or certain type of alarm may depend upon the state of available battery resources.

In step 130, detected seizure-related events may be classified as being associated with a particular type of physiological event or as a member of a group of physiological event types. In some embodiments, seizure-related events may be classified into one or more groups of events including, for example, non-seizure movements, GTC seizures, PNES events, complex-partial seizures, seizures that include a clonic phase, seizures that include a tonic phase, atonic seizures, and myoclonic seizures. In some embodiments, events may also be characterized based, for example, on intensity, duration, coherence, relationship to other physiological factors, other factors, and combinations thereof. By way of nonlimiting example, a relationship to other physiological factors may include whether a certain seizure-related event is correlated in some manner with normal or abnormal levels of activity measured using one or more sensors suitable for collecting EKG, EEG, temperature, acceleration, other data, or combinations thereof. In some embodiments, classification may be performed to a first classification level using one or more classification routines in order to generate classification data useful in automatic and/or active threshold adjustment. However, a more complete classification of events may be performed using one or more post-processing routines executed after a time or date of patient monitoring wherein seizure-related events were detected.

As already described herein, a number of different seizure-detection routines may be applied for classification of detected seizure-related events. For example, a number of seizure-detection routines were described as routines executable by classification module 94. And, in some embodiments, those routines may be applied, individually or in combination, to classify detected seizure-related events in the step 130.

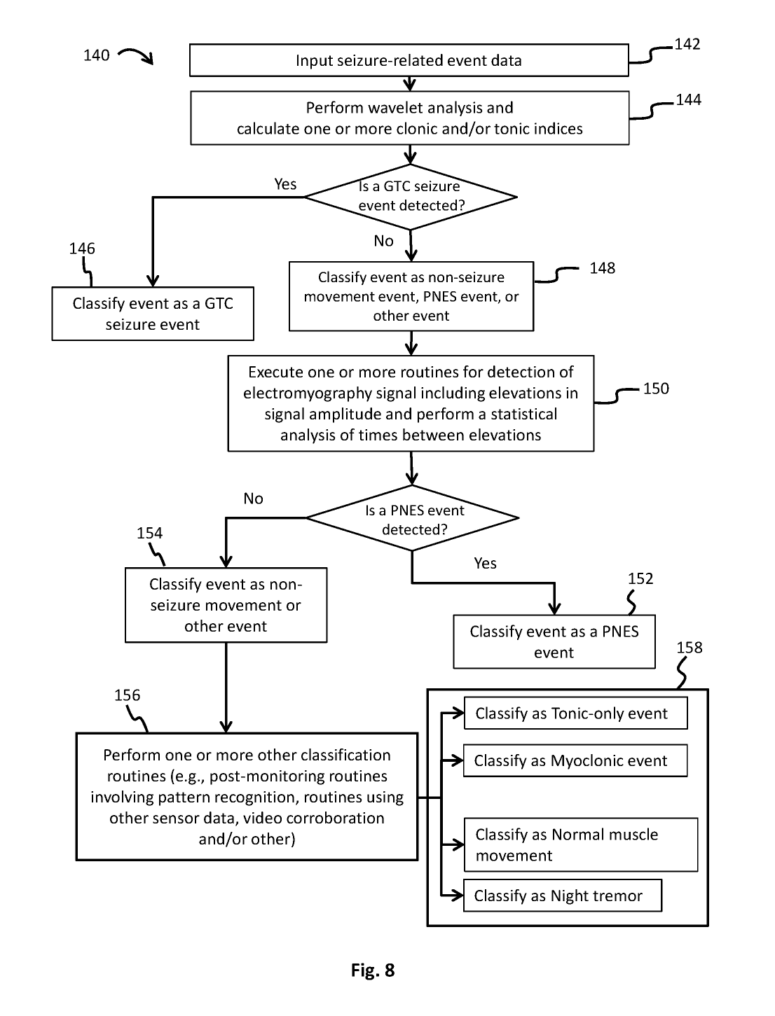

For example, in some embodiments, classification step 130 may include execution of one or more steps shown in a method 140 for classification of seizure-related events, as further described in relation to FIG. 8. As shown therein, in a step 142, seizure-related event data may be input or identified for processing. For example, seizure-related event data may be input in method 140 as part of a response to one or more seizure-related event detections in step 128, or EMG data may be input in some other way, such as based on another trigger. Any of various combinations of steps remaining in method 140 may then be executed as may be suitable for execution of different embodiments of seizure-related event classification. For example, a seizure-related event may be classified to various degrees or levels by sequential execution of steps 144, 150, and 156.

In step 144, wavelet analysis and calculation of one or more clonic and/or tonic indices may be performed on identified seizure-related event data. Based on that analysis, if a GTC seizure-related event is detected, the detected seizure-related event may be classified as a GTC seizure event (as shown in grouping 146). Also, wavelet analysis and calculation of one or more clonic and/or tonic indices may be particularly valuable as a method of classifying the duration of different phases of a seizure or of seizure duration as a whole. If a GTC seizure event is not detected or if positive detection of one or more PNES or non-seizure events is made, the seizure-related event data may be classified as a non-seizure movement, PNES event, or other event, as shown in grouping 148. Generally, seizure-related events classified in grouping 148 may present a lower risk of adverse effects to the patient than events classified in grouping 146.

In some embodiments, a seizure-related event classified in grouping 148 may be further classified using one or more additional seizure-detection routines. For example, as shown in step 150, a seizure-detection routine may analyze EMG data for detection of samples of electromyography signals including elevations in signal amplitude, and the routine may perform a statistical analysis of times between elevations. For example, as shown in a grouping 152, if the times between the elevations change in a manner that is typical of a non-epileptic psychogenic event, an event may be classified as a PNES event. For example, in some of the classification routines herein, the slope of a trend line for times between elevated portions of samples versus a detected sample number of less than about 0.8 may be used to classify an event as a PNES event. Further by way of example, as shown in a grouping 154, if no recognizable pattern of elevations is identified, events may be classified as non-seizure movements or as some other type of event.

In some embodiments, one or more other classification routines may be executed to further classify events in grouping 154. For example, any of various suitable routines may be executed in a step 156. Some of the routines which may be executed in step 156 may be executed as post-monitoring methods of analysis. That is, some of the routines executed in step 156 may be executed or results may be analyzed at some later time or date after a patient monitoring period in which a seizure-related event was detected. For example, in some embodiments, video or other suitable data may be reviewed and used to classify a detected seizure-related event as being one of a tonic-only, myoclonic, normal muscle movement, night tremor, or other physiological event as shown in grouping 158.

In some embodiments, classification data derived from the classification of seizure-related events in step 130, such as may be achieved using exemplary classification method 140 or other classification methods herein, may be communicated to one or more processors, including, for example, one or more processors included in threshold adjustment module 96. In some embodiments, classified seizure-related event data may be communicated to one or more processors configured to organize one or more alarm responses. For example, information concerning classification of one or more seizure-related events may be communicated to alarm initiation module 98. Accordingly, active alarms may be transmitted in order to provide medical care to a patient should the patient experience a seizure-related event and if, for example, that event is of a type warranting a certain type of alarm response. In some embodiments, including, for example, if an alarm or other response may have been issued prior to seizure-related event classification, an alarm or other response may be appropriately updated based on event classification. For example, if appropriate, a queued warning alarm may be canceled. Or, a caregiver may be given an option to cancel or update an emergency alarm, based, for example, on classification data or results of a classification analysis.

In a step 132, a processor, such as, for example, a processor that may be included in threshold adjustment module 96, may evaluate or score how well one or more detection conditions may function in identifying one or more types of seizure-related events when using one or more thresholds or groups of thresholds. In some embodiments, step 132 may operate at regular intervals or when some convenient number of one or more seizure-related events are classified. For example, whenever some batch of classified seizure-related events becomes available, scoring or evaluation of one or more detection conditions may execute. In some embodiments, evaluation or scoring of detection conditions may automatically be executed whenever a detection unit 32 is recharging or when a hard wire is provided between detection unit 32 and a base station processor 34. In some embodiments, evaluation or scoring of detection conditions may automatically be executed whenever a false positive event is identified.

A processor configured for executing step 132 may receive various data in order to score or evaluate routines and/or thresholds for seizure detection. For example, a processor included in threshold adjustment module 96 may receive data including, for example, classification data from step 130 and data associated with threshold settings that were used in step 126 to identify detected and now classified seizure-related events. In some embodiments, threshold adjustment module 96 may receive raw or processed data from a portion of EMG data, including, for example, one or more portions of EMG data associated with one or more detected and/or classified seizure-related events. Furthermore, in some embodiments, threshold adjustment module 96 may be capable of evaluating raw or processed data to determine if any of various threshold settings, if used in one or more seizure-detection routines, may have successfully detected seizure-related muscle activity in a portion of EMG data. For example, threshold adjustment module 96 may evaluate if one or more physiological events were accurately identified, if data from non-seizure periods may have triggered a false positive event, or both. Thus, in some embodiments, threshold adjustment module 96 may be capable of evaluating settings and/or routines not previously applied in step 126.

In some embodiments, threshold adjustment module 96 may receive information about one or more measured properties detected for a seizure-related event and/or may receive other information suitable to determine if any of various threshold settings, if used in one or more seizure-detection routines, may have successfully detected seizure-related muscle activity in a given portion of EMG data. Thus, again, in some embodiments, threshold adjustment module 96 may be capable of evaluating settings and/or routines not previously applied in the collection step 126.

In some embodiments, such as in step 126, in addition to evaluating whether one or more seizure-detection routines identified a seizure-related event and issuing a response to such a detection, identification module 92 may also evaluate results of whether one or more other seizure-detection routines, such as one or more routines with higher or lesser sensitive thresholds, would have detected seizure-related muscle activity in a certain portion of EMG signal. The results of whether seizure-related muscle activity would have been detected may then be sent to threshold adjustment module 96. Thus, again, in some embodiments, evaluation of seizure-detection routines not actively used for patient monitoring in the collection step 126 may also be evaluated or scored.

In some embodiments, a number of performance metrics may be applied for evaluating one or more seizure-detection routines and/or one or more thresholds or groups of thresholds in step 132. For example, in some embodiments, any of the performance metrics including selectivity, group sensitivity, patient sensitivity, rate of detection, rate of false positive detection, battery lifetime or performance when using the one or more seizure-detection routine, duty cycle of one or more seizure-detection routine, other suitable metrics, and combinations thereof may be determined. A performance metric may be determined for a single seizure-detection routine when applying a number of different thresholds or groups of thresholds. Thus, use of different thresholds or groups of thresholds may be compared. For example, based on that comparison, a preferred threshold or group of thresholds for a seizure-detection routine may be determined. In addition, in some embodiments, performance metrics for different seizure-detection routines may be determined. Accordingly, different routines among one or more seizure-detection routines may be compared. For example, a processor, tasked with scoring or evaluating one or more seizure-detection routines and/or one or more thresholds or groups of thresholds, may create a matrix of data wherein variables, including, for example, each of a type of seizure-detection routine and value of one or more threshold settings, are evaluated and compared with respect to one or more of the aforementioned performance metrics or other suitable metrics.

In some embodiments, in step 132, a processor, such as, for example, a processor that may be included in threshold adjustment module 96, may evaluate either or both of a group and/or a patient sensitivity of detection for one or more seizure detection routines and/or one or more thresholds or groups of thresholds with respect to one or more seizure-related event types. As used herein, the group sensitivity for a detection condition (e.g., use of one or more seizure-detection routines and/or one or more threshold settings or group of settings) with respect to one or more types of seizure-related events refers to a proportion of seizure-related events of the one or more types that may be detected with respect to a reference set of EMG data for classified physiological events of the one or more types for a group of patients when using the detection condition. For example, if a reference set of EMG data for classified physiological events includes 100 events of a certain type and if 99 of those events would be detected when using a certain detection condition, such as the group sensitivity of the detection condition with respect to that type of event may be expressed as 0.99 or 99%. A reference set of classified physiological events may include EMG data derived from any number of patients.

Thus, for example, in the step 132, a first performance metric used to evaluate or score how well said a group of one or more seizure-detection routines function in detection of said one or more seizure-related events may evaluate a sensitivity for detection of GTC seizures using data derived from any number of patients. A second performance metric may include a rate of detection of non-seizure physiological events. For example, a rate of identification of seizure-related events that are initially detected in step 126 and later classified as non-seizure events in step 130 may be determined. Thus, for example, patient specific data may be used for determining one performance metric, but group data may be used for sensitivity of detection of GTC seizures. Advantageously, such an approach may allow for efficient adjustment of thresholds for a patient before a significant body of GTC seizures is collected for an individual patient.

As used herein, the patient sensitivity for a detection condition with respect to one or more types of seizure-related events refers to a proportion of seizure-related events of the one or more types that may be detected with respect to a patient-specific reference set of classified physiological events of the one or more types when using the detection condition. For example, if a patient-specific reference set of classified physiological events includes 50 events of a certain type and if 40 of those events would be detected when using a certain detection condition, the patient sensitivity of the detection condition with respect to that type of event may be expressed as 0.80 or 80%. A patient-specific reference set of classified physiological events may include data derived from a particular patient.

In some embodiments, in step 132, a processor, such as, for example, a processor that may be included in threshold adjustment module 96, may score or evaluate a group sensitivity and/or a patient sensitivity of a detection condition with respect to one or more seizure-related event types, including, for example, epileptic seizures, complex-partial seizures, GTC seizures, seizures that include one or more tonic or clonic phase portions, other types of seizure-related events, and combinations thereof. In some embodiments, sensitivity performance metrics may be optimized or maintained above some desired level for one or more detection conditions with respect to types of seizure-related events that are most likely to demand an immediate emergency response.

Various standards may be used to determine a reference set of classified physiological events and an associated group sensitivity of detection. For example, in one high or rigorous standard, data may be taken from all or some plurality of recorded patients, or patients of some demographic when monitored in a controlled setting. Seizure-related event classification may be verified using one or more sensors, including, for example, EMG sensors, and preferably including video monitoring. In this high or rigorous standard, seizure-related event detection and classification may be verified by trained medical persons such as doctors. Based on that rigorously collected and verified data, it may be empirically determined that, when using one or more seizure-detection routines and/or one or more thresholds or groups of thresholds, some proportion of seizure-related events of a certain physiological type will be detected for a group of patients. In some embodiments, seizure-related events characterized in this rigorous manner may be used or included in construction of a reference set of classified physiological events.

In some embodiments, other data may be used to construct a reference set of classified physiological events. For example, as discussed in relation to step 124, initial thresholds may sometimes be selected in order to provide high sensitivity for detection. Accordingly, a high proportion of significant seizure-related muscle activity may be detected in step 126 and classified in step 130. And, in some embodiments, classified seizure-related-event data collected from one or more patients in method 120 may be aggregated and included, individually or in combination with other suitable classified seizure-related-event data, in defining a reference set of classified physiological events.

In some embodiments, data associated with a reference set of classified physiological events may be included in a reference threshold data file. For example, in step 132, a processor, such as, for example, a processor that may be included in threshold adjustment module 96, may store or have access to the reference threshold data file. Based on that reference threshold data file, threshold adjustment module 96 may be configured to estimate that some proportion of events of a certain physiological type may be detected when using a certain seizure-detection routine when using one or more thresholds or groups of thresholds. For example, it may be determined that greater than 95%, greater than 99%, or an even higher proportion of GTC seizures, seizures including a clonic phase portion, or seizure-related events of some other type may be detected when using a certain set of thresholds in a certain seizure-detection routine.

Various standards may also be used to determine a patient-specific reference set of classified physiological events and an associated patient sensitivity of detection. For example, in one high or rigorous standard, data may be taken when a certain patient is monitored in a controlled setting. Seizure-related event classification may be verified using one or more sensors, including, for example, EMG sensors, and preferably including video monitoring. In this high or rigorous standard, seizure-related event detection and classification may be verified by trained medical persons such as doctors. Based on that rigorously collected and verified data, it may be empirically determined that, when using one or more seizure-detection routines and/or one or more thresholds or groups of thresholds, some proportion of seizure-related events of a certain physiological type will be detected for a certain patient. In some embodiments, seizure-related events characterized in this rigorous manner may be used or included in construction of a patient-specific reference set of classified physiological events.

In some embodiments, other data may be used to construct a patient-specific reference set of classified physiological events. For example, as discussed in relation to step 124, initial thresholds may sometimes be selected in order to provide high sensitivity for detection. Accordingly, a high proportion of significant seizure-related muscle activity may be detected in step 126 and classified in step 130. And, in some embodiments, patient-specific classified seizure-related-event data collected for a patient in method 120 may be aggregated and included, individually or in combination with other suitable classified seizure-related-event data, in defining a patient-specific reference set of classified physiological events.

Generally, while embodiments herein include some methods wherein a patient-specific reference set of classified physiological events may be determined in various ways, in some preferred embodiments, a patient-specific reference set of classified physiological events may be determined without needing to monitor a new patient in a controlled or hospital-like setting. For example, initial threshold settings applied for a new patient may be applied in step 124 based on empirical data from other patients, and that empirical data may also be used to determine a reference set of classified physiological events. Other steps or iterations of steps following initial threshold selection in step 124 may be executed to collect patient-specific data. As a suitable number of patient-specific seizure-related events are detected, such as a number to arrive at a statistically significant sample set, threshold adjustment module 96 may transition from scoring one or more detection conditions based on a reference set of classified physiological events to scoring based on a patient-specific reference set of classified physiological events. For example, a transition between those performance standards may be based on whether a threshold number of GTC seizures is recorded for a patient. For example, in step 132 or in some initial iterations of step 132, identification module 92 may initially choose or select detection conditions based solely or predominantly on a group sensitivity of detection, but as more data is collected and patient-specific seizure-related events are detected, the system may adjust to evaluate detection conditions based solely or predominantly on a patient sensitivity of detection.

In some embodiments, data suitable to calculate a patient sensitivity performance metric may be collected in one or more iterations of execution of method 120. For example, in step 126 one or more seizure-related events may be detected, and those events may be classified in step 130. Thus, in some embodiments, patient-specific seizure-related event data may be collected during execution of method 120. In some embodiments, initial threshold settings selected in step 124 may be suitable to detect all significant seizure-related muscle activity and pass associated seizure-related events forward to classification. For example, in some embodiments, initial threshold settings may themselves be based on empirical data for a group of patients and may generally be selected to apply only a weak or nonselective filter to detected seizure-related muscle activity. And, it may therefore be assumed that all normally presenting physiological events of a certain type would be detected and thus subject to classification. Of course, if a patient or other caregiver identifies that one or more physiological events were missed by the method 120, they may report the missed physiological event.

In some embodiments, in step 132, a processor, such as, for example, a processor that may be included in threshold adjustment module 96, may evaluate a selectivity of detection for a specific seizure-related event type when using one or more seizure detection routines and/or one or more thresholds or groups of thresholds. As used herein, the selectivity of detection for a detection condition with respect to one or more types of seizure-related events refers to a proportion of seizure-related events of the one or more types that may be detected with respect to all detected seizure-related events when using the detection condition. For example, if 50 total seizure-related events are detected when using a certain detection condition, but only 2 of the total detected seizure-related events are GTC seizure events, the selectivity of the detection condition for GTC seizure events may be expressed as 0.04 or 4%.

In some embodiments, in step 132, a processor may evaluate or score how well one or more seizure-detection routines may function in identifying seizure-related events that demand an emergency response or other immediate response as opposed to other seizure-related events including those that may not demand an immediate response. For example, for some patients or patients in some selectable states, it may be deemed that events classified as GTC seizure events may demand an emergency response whereas other events, such as non-seizure movements, may preferably be ignored or only logged or stored for post-process review. Accordingly, it may be desirable that primarily GTC seizure events are identified in step 126. For example, it may be desirable to detect GTC seizure events with high selectivity. Notably, by enhancing selectivity for detection of GTC seizure events in step 126, the duty cycle of operation for classification module 94, or other suitable processors used in step 130, may be decreased. Thus, in some embodiments, battery resources may be conserved as selectivity for GTC seizure events improves. In some embodiments, optimization of detection conditions may include adjusting detection conditions subject to a requirement that a selected detection condition maintains a sensitivity above a desired sensitivity level. However, detection conditions that both maintain a desired sensitivity level and achieve a desired selectivity for detection of GTC seizures may be preferred or scored higher than other detection conditions with lower selectivity for GTC seizures.

In some embodiments, a processor executing step 132 may also score or estimate how well one or more seizure-detection routines may function in excluding one or more types of events when using one or more thresholds or groups of thresholds. To estimate how well one or more seizure-detection routines may function in excluding one or more types of seizure-related events, a rate of detection for seizure-related events of a certain seizure-related event type may be determined when using one or more thresholds or groups of thresholds. For example, a rate of detection of non-seizure movements may be determined. Generally, to enhance battery lifetime, it may be desired that a low rate of detection of non-seizure movements may be achieved. In some embodiments, optimization of detection conditions may include adjusting detection conditions subject to a requirement that a selected detection condition maintains a sensitivity above a desired sensitivity level. However, detection conditions that both maintain a desired sensitivity level and minimize a rate of detection of non-seizure movements may be preferred or scored higher than other detection conditions with higher rates of detection of non-seizure movements.

Various embodiments for scoring or evaluating data in step 132 may be applied in the systems and methods herein. In some embodiments, evaluating how well one or more seizure-detection routines function in identifying seizure-related events or seizure-related events of one or more event types may include determining if some number of false positive detections were made. For example, a false positive detection may be logged when a seizure-event was identified using one or more seizure-detection routine in the module 92, but the classification module 94 determines that the event should be classified as a non-seizure movement. In some embodiments, a processor, such as a base station or detection unit processor, may further provide input capability for a patient to input data for or false positive detections or missed seizures. Or, false positive detections or missed seizures may be logged in some other way, such as by a caregiver, when those seizure events are reported. Thus, in some embodiments, methods and systems herein may automatically adjust thresholds without receiving data from one or more persons. However, some methods and systems may alternatively be prompted to adjust threshold settings when receiving data from one or more persons qualified to input seizure-related event data. Accordingly, in some embodiments, evaluation of how well one or more seizure-detection routines may function in identifying seizure-related events may include determining if some number of false positive detections were made, determining if one or more GTC seizures were missed, or a combination of both. And, in some embodiments, systems and methods herein may automatically increase one or more threshold settings when a certain number of false positive detections are made. Or, a seizure-detection routine may attempt to reduce false positive detections by adjusting a threshold setting in some other way. Likewise, in some embodiments, systems and methods herein may decrease one or more threshold settings when a missed seizure is logged. Or, a seizure-detection routine may attempt to increase sensitivity for detection of GTC seizures in some other way.

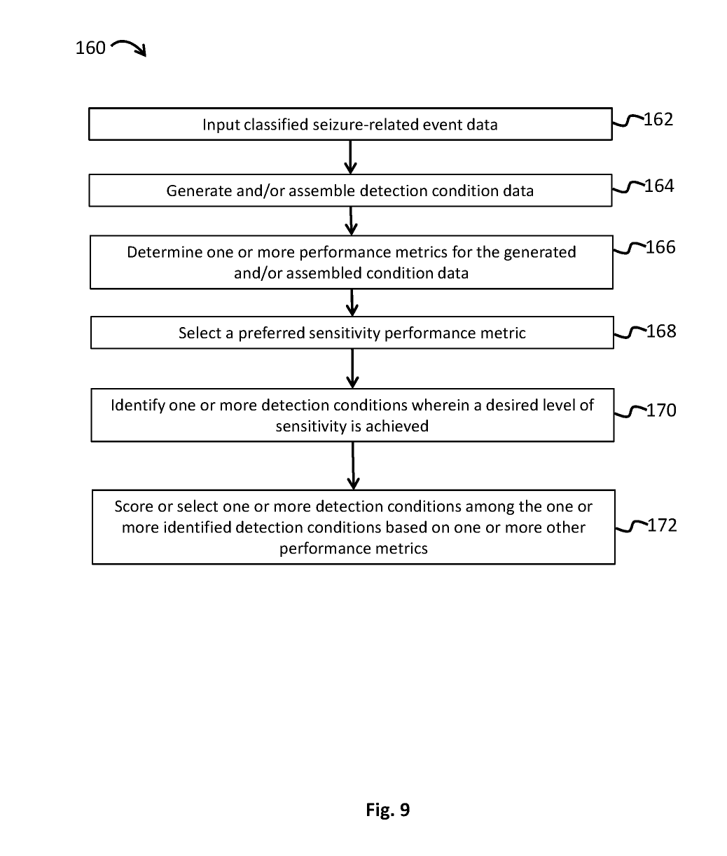

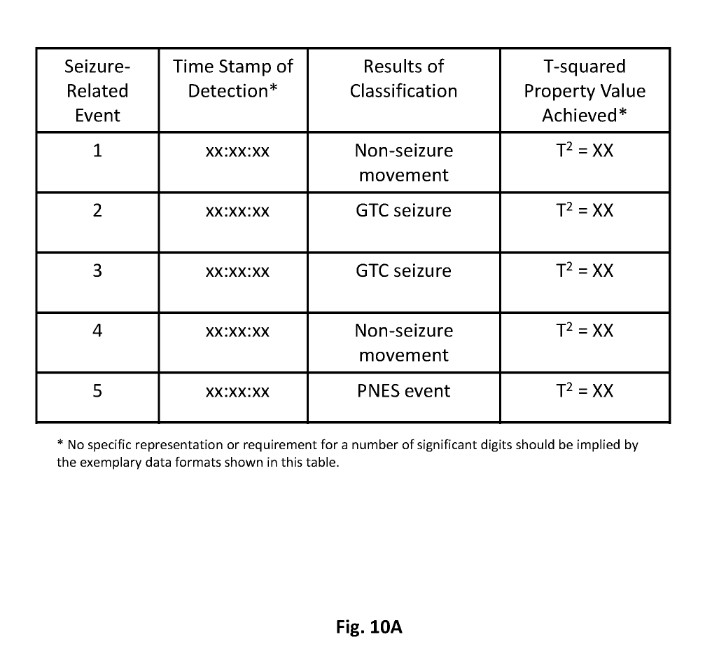

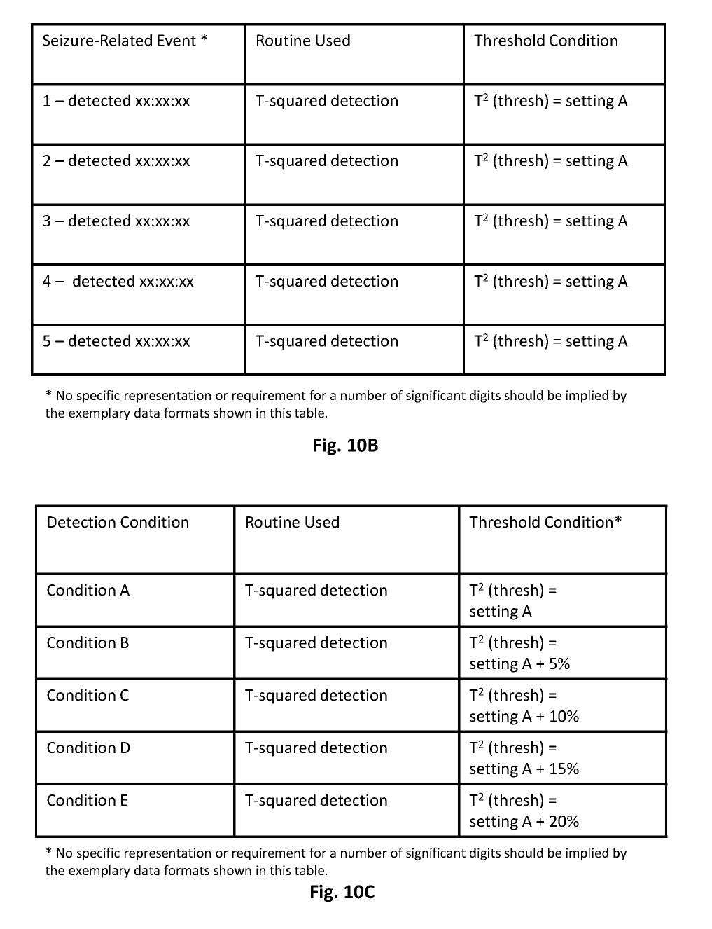

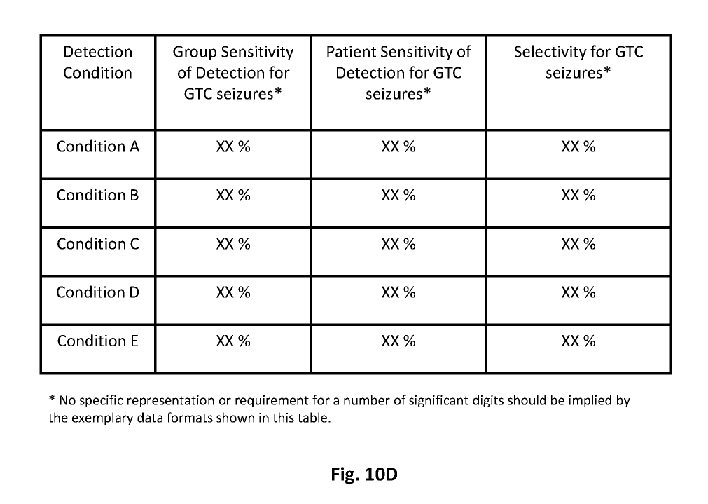

In some embodiments, step 132 may include execution of a method 160 for scoring classified seizure-related event data, as is further described in relation to FIG. 9. As shown therein, in a step 162, classified seizure-related event data may be input for evaluation or scoring. For example, classified seizure-related event data may be selected for use by one or more processors that may be included in threshold adjustment module 96. Exemplary classified seizure-related event data is also shown in FIG. 10A. As shown therein, classified seizure-related event data may be time stamped, and other information, such as, for example, one or more property values achieved, may be input or otherwise made available to threshold adjustment module 96.

In step 164, detection condition data may be assembled and/or generated. For example, assembling of detection condition data may include accessing data for detected seizure-related events (e.g., as may be detected in one or more iterations of step 126) and the detection conditions used to detect the detected seizure-related events. For example, FIG. 10 B shows model data wherein the time-stamped seizure-related events in FIG. 10A are identified as associated with a detection condition including use of a T-squared detection routine configured to compare a detected T-squared value to a threshold T-squared value (designated as setting A in FIG. 10B).

In some embodiments, generation of detection condition data may include constructing one or more detection conditions, including, for example, one or more detection conditions not previously used in one or more iterations of step 126. For example, in some embodiments, detection conditions may be generated wherein one or more thresholds may be raised or otherwise adjusted to generally decrease an expected number of detected seizure-related events. For example, a threshold T-squared value may be raised, which generally may decrease the sensitivity for detection of seizure-related events when using a T-squared based seizure-detection routine. For example, a threshold T-squared value may be raised in increments of about 5% or some other suitable value in order to generate a range or set of detection conditions. For example, in FIG. 10C, a first detection condition (condition A) is listed showing the detection condition applied to detect the exemplary events in FIG. 10B. In addition, several different detection conditions are generated by raising the threshold T-squared setting above the setting used in the first detection condition.

In step 166, one or more performance metrics may be determined for generated and/or assembled detection condition data. In some embodiments, threshold adjustment module 96 may receive raw or processed data or other information suitable to test condition data not previously applied in step 126. For example, as shown in FIG. 10A, one or more property values achieved may be made available to threshold adjustment module 96, and a processor included in threshold adjustment module 96 may include instructions suitable to compare the property values to thresholds in order to determine whether an event may be detected for any of a number of different threshold values. Accordingly, performance metrics may be calculated for either or both of generated and/or assembled detection condition data. For example, FIG. 10D shows some exemplary performance metrics that may be calculated for the detection conditions shown in FIG. 10C.

In some embodiments, threshold adjustment module 96 may be configured to select either of a group sensitivity, patient sensitivity, and/or combination thereof for evaluating the performance of detection condition data. For example, as shown in step 168, a preferred sensitivity performance metric may be selected. For example, a decision may be made on whether to evaluate sensitivity based on a group and/or a patient-specific standard. In some embodiments, deciding whether either of a group sensitivity or patient sensitivity performance metric is selected may be based on whether a statistically significant number of patient-specific seizure-related events or patient-specific seizure-related events of a certain type have been detected. For example, in some embodiments, if greater than about 10, greater than about 15, or greater than about 20 GTC seizure events have been detected for a patient, a patient sensitivity performance metric may be selected. In some embodiments, a combination of patient sensitivity and group sensitivity performance metrics may be selected. For example, the aforementioned sensitivity metrics may be weighted with relative weights based on the availability of patient-specific seizure-related event data.

In step 170, one or more detection conditions may be identified wherein a desired level of sensitivity is achieved. For example, it may be found that, when using a seizure-detection routine that includes determining a T-squared value, a desired level of sensitivity for detection of GTC seizures is achieved for each of a number of different T-squared threshold levels. Accordingly, each of the one or more detection conditions that meet the desired level of sensitivity may be further evaluated, whereas other detection conditions that do not meet the desired level of sensitivity may be deemed unacceptable.

In step 172, one or more detection conditions among the one or more detection conditions identified in step 168 may be scored or selected based on one or more other performance metrics. For example, in some embodiments, the one or more other performance metrics may be selected from selectivity, rate of detection, battery performance, other suitable metrics, and combinations thereof. For example, in some embodiments, detection conditions that both maintain a desired sensitivity level and achieve a desired selectivity for detection of GTC seizures may be preferred or scored higher than other detection conditions with lower selectivity for GTC seizures. For example, referring to the model data shown in FIGS. 10A-10D, it may be found that each of detection conditions A-C maintains a desired level of sensitivity. However, it may be found that, when using the higher T-squared threshold associated with detection condition C, selectivity for GTC seizures is improved. Accordingly, detection condition C may be selected.

Referring back to method 120 in FIG. 7, in some embodiments, once a detection condition is selected, instructions may be sent (e.g., as shown in the step 134) to update threshold settings and/or other detection condition settings which may be applied in a next iteration or loop of method 120. For example, referring to the model data shown in FIGS. 10A-10D, the higher T-squared thresholds associated with detection condition C may be selected, and as described in step 134, instructions may be sent to identification module 92 instructing the module to adopt the higher T-squared threshold setting. That is, in step 124, the higher T-squared thresholds associated with detection condition C may be selected. Additional EMG signal may then be collected in step 126 using the updated or adjusted threshold setting. In some embodiments, method 120 may operate for any number of loops. And, for example, threshold settings may be adjusted or optimized continuously throughout patient monitoring. In some embodiments, with reference to step 133, if the system does not determine detection conditions indicative of a certain performance, the method 120 may send instructions to update threshold settings and continue to monitor the patient and collect data and score or evaluate the system as indicated at 134. Alternatively, in some embodiments, irrespective of whether a desired performance is achieved, the system may periodically update thresholds if improved detection conditions are identified.

In some embodiments, once one or more detection conditions are identified that achieve a desired performance, a detection system may be deemed calibrated, and as shown in the step 136, the system may exit method 120. For example, as described in step 133, if the system has achieved one or more desired scores suitable to indicate a desired detection performance, the system may exit method 120, as shown in step 136. In some embodiments, when exiting method 120, as shown in step 136, a message may be sent to a patient indicating that the system is fully calibrated or calibrated to a desired level. For example, a calibrated system may achieve each of a desired sensitivity for detection of epileptic seizures and a desired selectivity for detection of epileptic seizures, or other performance metrics may be met.

In some embodiments, threshold adjustment module 96 may receive data for seizure-related events classified as true GTC seizures and data for seizure-related events classified as being associated with some other type of seizure-related event. For example, threshold adjustment module 96 may receive data for seizure-related events classified as true GTC seizures and data for other seizure-related events that were classified. In some embodiments, threshold adjustment module 96 may also receive a more detailed classification of events other than those classified as a true GTC seizure. For example, in embodiments where step 130 includes execution of classification method 140, threshold adjustment module 96 may receive information about events classified in any of the steps 144, 150, and 156. For example, threshold adjustment module 96 may receive data that seizure-related events were grouped as shown in any of the groupings 146, 148, 152, 154, and 158 or combinations thereof.

In some embodiments, threshold adjustment module 96 may periodically, such as at some regular or suitable intervals, evaluate whether a rate of detection of non-seizure movements or non-seizure movements and other events different than GTC seizure events may be higher than desired. For example, threshold adjustment module 96 may identify that, when using one or more threshold settings, greater than about 2 to about 8 non-seizure movement events or other events different than GTC seizure events are detected per hour. This rate may be deemed unacceptably high. For example, because classification module 94 may be initiated when these events are detected, a duty cycle of operation for classification module 94 may be inordinately high and battery resources may be compromised.

Accordingly, in step 132, threshold adjustment module 96 may evaluate whether changing one or more threshold settings may decrease a rate of detection for one or more events. In some preferred embodiments, threshold adjustment module 96 may evaluate whether an adjustment of settings within acceptable boundaries may decrease a rate of detection for one or more event types, but maintain a desired sensitivity for detection of one or more other event types. For example, threshold adjustment module 96 may attempt to find threshold settings wherein a rate of detection of non-seizure events is decreased, but where sensitivity for detection of GTC seizures or some other event is maintained within acceptable boundaries. In some embodiments, acceptable boundaries for threshold setting adjustment may be set based on a calculation or estimation of boundaries that maintain a desired level of sensitivity for detection of one or more event types. If threshold adjustment module 96 determines that it may apply new threshold settings or adjusted settings in order to decrease a rate of detection of non-seizure events, but maintain a desired level of sensitivity, the threshold adjustment module 96 may determine how to adjust detection conditions in order to apply the new threshold settings. For example, threshold adjustment module 96 may send instructions to apply the new threshold settings in a step 134.

In some embodiments, threshold adjustment module 96 may be configured to use any of various standards to determine if all or some proportion of one or more types of events may be detected when using one or more thresholds or groups of thresholds. For example, threshold adjustment module 96 may be configured to apply a rigorous standard based on data taken from one or more patients in a controlled setting and wherein event classification is verified by trained professionals, another standard based on customized or individual patient data collected during one or more monitoring sessions for the subject patient, one or more other standards, or combinations thereof.

In some embodiments, threshold adjustment module 96 may include rules to switch between standards for evaluating whether all or some proportion of one or more types of events may be detected when using one or more thresholds or groups of thresholds. For example, a first standard may be a rigorous standard based on data taken from one or more patients in a controlled setting and wherein event classification is verified by trained professionals, or some other suitable standard may be applied. A second standard may be based on customized or patient-specific event data. Threshold adjustment module 96 may be configured to apply the first standard while it is collecting customized or patient-specific event data. When some number of suitable events are detected for a patient, threshold adjustment module 96 may switch over to the second standard. For example, once a statistically significant number of events are collected for a patient, threshold adjustment module 96 may move to automatically apply patient-specific data.

In some embodiments, including, for example, when a patient has just started a monitoring regimen or before times when the system 90 has collected significant data for that patient, threshold settings may be set to only provide a filter with limited selectivity in discriminating detected seizure-related muscle activity that corresponds to true seizures from non-seizure movements. Accordingly, a relatively large proportion of elevated muscle movements may trigger execution of one or more seizure-detection routines using classification module 94. Thus, a duty cycle of operation for seizure-detection routines executed using classification module 94 may be relatively high. For example, in some embodiments, classification module 94 may, under some conditions, including, for example, when fully calibrated, operate with a duty cycle of operation of less than about 1:10 or less than about 1:100. However, in some embodiments, during initial setup of the system 90 or during training or calibration, a duty cycle of operation for classification module 94 may be as high as about 1:2.5 or as high as about 1:5. Accordingly, battery life may, at least in part, be limited during training or calibration because of a frequency of execution of classification module 94.

In some embodiments, a patient may be instructed that, when being monitored using system 90 for the first time or during a training or reference period, the battery life of the detection unit 32 may be limited. For example, the battery may only last about 90%, about 75%, about 50%, or some other percentage factor as long as it would otherwise be expected to operate during normal operation. For example, in some embodiments, a detection unit 32 may typically operate without needing to be recharged for up to about 12 to about 36 hours. However, during a training or reference period, a battery may last only for about 75% or some other percentage value of that time. In some embodiments, a patient may be advised that, when wearing a detection unit 32 during a training or reference period, they may choose to have access to a spare unit, or they can recharge the unit after an appropriate time period. In other embodiments, the battery lifetime of a device may be only minimally changed during initial wearing of the device or during a training or reference period. And, in some embodiments, when a detection unit is calibrated, a duty cycle of operation of one or more seizure detection suitable for classification of detected seizures events may be less than a maximum threshold duty cycle, the maximum threshold duty cycle established so that battery life is at least 50%, at least 75%, at least 90%, or at least 95% of a battery life for the detection unit without execution of the one or more seizure detection suitable for classification of detected seizures events.

In some embodiments, systems herein may be configured to execute responses in a manner that may depend upon whether the system is in one of several selectable states. For example, a patient may be given an option to select one or more different monitoring settings based, for example, on whether the patient is in bed sleeping, whether the patient is at home alone, or if the patient is at home with another person. And, depending on whether the patient is in one or another of those states, a monitoring system may select a response protocol that may include either a warning transmission protocol or emergency transmission protocol. For example, it may be deemed, at least for some seizure-related events for some patients, that a warning protocol may be initiated based on detection of an event in the identification module (92), when the patient is sleeping or in some other state. And, an emergency response may not be initiated until the seizure is verified in the classification module (94) or until a classified seizure is deemed to present certain risk for adverse effects of a seizure. For example, in some embodiments, an intensity and/or duration of one or more phases of a GTC seizure may be classified and used to determine that an emergency response should be initiated.

Generally, the devices of a seizure detection system may be of any suitable type and configuration to accomplish one or more of the methods and goals disclosed herein. For example, a server may comprise one or more computers or programs that respond to commands or requests from one or more other computers or programs, or clients. The client devices may comprise one or more computers or programs that issue commands or requests for service provided by one or more other computers or programs, or servers. The various devices in FIG. 2, e.g., 32, 34, 40, 42, 44 and/or 52, may be servers or clients depending on their function and configuration. Servers and/or clients may variously be or reside on, for example, mainframe computers, desktop computers, PDAs, smartphones, tablet devices, netbooks, portable computers, portable media players with network communication capabilities, cameras with network communication capabilities, smartwatches, wearable computers, smart sensors, and the like.

A computer may be any device capable of accepting input, processing the input according to a program, and producing output. A computer may comprise, for example, a processor, memory and network connection capability. Computers may be of a variety of classes, such as supercomputers, mainframes, workstations, microcomputers, PDAs and smartphones, according to the computer's size, speed, cost and abilities. Computers may be stationary or portable and may be programmed for a variety of functions, such as cellular telephony, media recordation and playback, data transfer, web browsing, data processing, data query, process automation, video conferencing, artificial intelligence, and much more.

A program may comprise any sequence of instructions, such as an algorithm, whether in a form that can be executed by a computer (object code), in a form that can be read by humans (source code), or otherwise. A program may comprise or call one or more data structures and variables. A program may be embodied in hardware or software or a combination thereof. A program may be created using any suitable programming language, such as C, C++, Java, Perl, PHP, Ruby, SQL, and others. Computer software may comprise one or more programs and related data. Examples of computer software include system software (such as operating system software, device drivers and utilities), middleware (such as web servers, data access software and enterprise messaging software), application software (such as databases, video software and media players), firmware (such as device-specific software installed on calculators, keyboards and mobile phones), and programming tools (such as debuggers, compilers and text editors).

Memory may comprise any computer-readable medium in which information can be temporarily or permanently stored and retrieved. Examples of memory include various types of RAM and ROM, such as SRAM, DRAM, Z-RAM, flash, optical disks, magnetic tape, punch cards, and EEPROM. Memory may be virtualized and may be provided in or across one or more devices and/or geographic locations, such as RAID technology. An I/O device may comprise any hardware that can be used to provide information to and/or receive information from a computer. Exemplary I/O devices include disk drives, keyboards, video display screens, mouse pointers, printers, card readers, scanners (such as barcode, fingerprint, iris, QR code, and other types of scanners), RFID devices, tape drives, touch screens, cameras, movement sensors, network cards, storage devices, microphones, audio speakers, styli and transducers, and associated interfaces and drivers.

A network may comprise a cellular network, the Internet, intranet, local area network (LAN), wide area network (WAN), Metropolitan Area Network (MAN), other types of area networks, cable television network, satellite network, telephone network, public networks, private networks, wired or wireless networks, virtual, switched, routed, fully connected, and any combination or subnetwork thereof. The network may use a variety of network devices, such as routers, bridges, switches, hubs, repeaters, converters, receivers, proxies, firewalls, translators and the like. Network connections may be wired or wireless and may use multiplexers, network interface cards, modems, IDSN terminal adapters, line drivers, and the like. The network may comprise any suitable topology, such as point-to-point, bus, star, tree, mesh, ring, and any combination or hybrid thereof.

Wireless technology may take many forms, such as person-to-person wireless, person-to-stationary receiving device, person-to-a-remote alerting device using one or more of the available wireless technology such as ISM band devices, WiFi, Bluetooth, cell phone SMS, cellular (CDMA2000, WCDMA, etc.), WiMAX, WLAN, and the like.

Communication in and among computers, I/O devices and network devices may be accomplished using a variety of protocols. Protocols may include, for example, signaling, error detection and correction, data formatting and address mapping. For example, protocols may be provided according to the seven-layer Open Systems Interconnection model (OSI model), or the TCP/IP model.

The systems and methods herein may thus be variously embodied as described in the following clauses:

1. An EMG detection system configured for automatic calibration of threshold settings for monitoring a patient for detection of seizure activity, the system comprising:
a wireless EMG detection unit (32), said wireless EMG detection unit including one or more EMG electrodes (54), the one or more EMG electrodes configured to collect an EMG signal from a patient, the wireless EMG detection unit (32) configured to remotely communicate with one or more caregiver devices (42, 44);
an identification module (92), said identification module including a processor configured to execute a first group of one or more seizure-detection routines for determining one or more property values of said EMG signal and comparing said one or more property values to one or more initial thresholds for detection of one or more seizure-related events;
a classification module (94), said classification module including a processor configured to execute a second group of one or more seizure-detection routines for classifying individual seizure-related events as being associated with one or more physiological activity types in order to provide classification data, the one or more physiological activity types including a generalized tonic-clonic seizure type and at least one other physiological activity type;
a threshold adjustment module (96), said threshold adjustment module including a processor configured to access said classification data and use said classification data to evaluate one or more performance metrics for said one or more seizure-detection routines when the routines are applying said one or more initial thresholds, the one or more performance metrics including a sensitivity for detection of generalized tonic-clonic seizures and a selectivity for detection of generalized tonic-clonic seizures, the threshold adjustment module configured to automatically adjust said one or more initial thresholds based on said one or more performance metrics in order to calibrate said EMG detection unit.

2. The system of clause 1 further configured to initiate one or more alarms based on the detection of said one or more seizure-related events.

3. The system of clause 1, said sensitivity for detection of generalized tonic-clonic seizures comprising either of a group sensitivity for detection of generalized tonic-clonic seizures or a patient-specific sensitivity for detection of generalized tonic-clonic seizures; and
said threshold adjustment module configured to select either of said group sensitivity for detection of generalized tonic-clonic seizures or said patient-specific sensitivity for detection of generalized tonic-clonic seizures based on a number of classified generalized tonic-clonic seizures for said patient.

4. The system of clause 1, said threshold adjustment module configured to determine a sensitivity for detection of generalized tonic-clonic seizures based on a weighted contribution of group sensitivity and patient-specific sensitivity.

5. The system of clause 1, said performance metrics further including a duty cycle for at least one seizure-detection routine among said second group of one or more seizure-detection routines.

6. The system of clause 1, said one or more property values including one or more of an amplitude value, a T-squared statistical value, a principal component value, a number of zero crossings, and combinations thereof.

7. The system of clause 1, said second group of one or more seizure-detection routines including one or more seizure-detection routine configured to detect samples of said EMG signal which include a peak and determine whether said samples show a pattern indicating the presence of a PNES event.

8. The system of clause 1, said second group of one or more seizure-detection routines including one or more seizure-detection routines configured to execute a wavelet transform in order to organize the EMG signal into a high frequency band of EMG signal and a low frequency band of EMG signal, and analyze said high frequency band of EMG signals and said low frequency band of EMG signals in order to determine whether a tonic phase and/or a clonic phase of a generalized tonic-clonic seizure is present.

9. The system of clause 1, said threshold adjustment module further configured to select one or more seizure detection routines among said first group of one or more seizure-detection routines for use in said EMG detection unit when the EMG detection unit is calibrated, the selection based on said one or more performance metrics.

10. The system of clause 1 wherein said identification module is included in said EMG detection unit and said classification module is physically separated from said detection unit.

11. The system of clause 1 wherein each of said identification module and said classification module are included in said EMG detection unit.

12. A method of calibrating an EMG system for monitoring a patient for seizure activity, the method comprising:
disposing an EMG detection unit including one or more EMG electrodes in association with one or more patient muscles, said one or more EMG electrodes configured for collecting an EMG signal in a form substantially representing seizure-related muscle activity;
collecting said EMG signal using said one or more EMG electrodes;
processing said EMG signal using a first group of one or more seizure-detection routines, the one or more seizure-detection routines configured for determining one or more property values of said EMG signal and comparing said one or more property values to one or more initial threshold in order to detect one or more seizure-related events;
classifying said one or more seizure-related events using a second group of one or more additional seizure-detection routines, said one or more additional seizure-detection routines configured to determine how individual seizure-related events relate to one or more physiological activity types, the one or more physiological activity types including a generalized tonic-clonic seizure type and at least one other physiological activity type;
evaluating how well said first group of one or more seizure-detection routines functions in detecting said one or more seizure-related events based on one or more performance metrics for said one or more seizure-detection routines when said one or more seizure-detection routines apply said one or more initial thresholds, the one or more performance metrics including a sensitivity for detection of generalized tonic-clonic seizures and a selectivity for detection of generalized tonic-clonic seizure; and
updating said one or more initial thresholds based on the evaluation of said one or more performance metrics in order to calibrate said EMG detection unit.

13. The method of clause 12 further comprising initiating one or more alarms based on the detection of said one or more seizure-related events.

14. The method of clause 12 wherein said sensitivity for detection of generalized tonic-clonic seizures comprises either of a group sensitivity for detection of generalized tonic clonic seizures or a patient-specific sensitivity for detection of generalized tonic-clonic seizures and further comprising:
selecting either of said group sensitivity for detection of generalized tonic-clonic seizures or said patient-specific sensitivity for detection of generalized tonic-clonic seizures based on a number of classified generalized tonic-clonic seizures for said patient.

15. The method of clause 12, said sensitivity for detection of generalized tonic-clonic seizures includes a weighted contribution of group sensitivity and patient-specific sensitivity.

16. The method of clause 12, said performance metrics further including a duty cycle of at least one seizure-detection routine among said second group of one or more additional seizure-detection routines.

17. The method of clause 16 wherein a maximum duty cycle of said at least one seizure-detection routine among said second group of one or more additional seizure-detection routines is targeted so that a battery lifetime is at least about 50% of a battery lifetime that may otherwise be achieved without execution of said at least one seizure-detection routine among said second group of one or more additional seizure-detection routines.

18. The method of clause 12, said one or more property values including one or more of an amplitude, a T-squared statistical value, a principal component value, a number of zero crossings, and combinations thereof.

19. The method of clause 12, said second group of one or more seizure-detection routines including one or more seizure-detection routine configured to detect samples of the EMG signal which include a peak and determine if said samples show a pattern indicating the presence of a PNES event.

20. The method of clause 12, said second group of one or more seizure-detection routines including one or more seizure-detection routine configured to execute a wavelet transform in order to organize EMG signal into a high frequency band of EMG signal and a low frequency band of EMG signal, and analyze said high frequency band of EMG signals and said low frequency band of EMG signals in order to determine whether a tonic phase and/or a clonic phase of a generalized tonic-clonic seizure is present.

21. The method of clause 12 further comprising selection of one or more seizure detection routines among said first group of one or more seizure-detection routines for use in said EMG detection unit when the EMG detection unit is calibrated, said selection based on said one or more performance metrics.

22. An EMG detection system for monitoring of a patient for detection of seizure activity, the system comprising:
a wireless EMG detection unit, said wireless detection unit including one or more EMG electrodes, the one or more EMG electrodes configured to collect an EMG signal for a patient substantially continuously over time;
wherein said detection unit is configured for remote communication with one or more caregiver devices;
an identification module including a processor configured to execute a first group of one or more seizure-detection routines for determining one or more property values of said EMG signal and comparing said one or more property values to one or more initial thresholds for detection of one or more seizure-related events;
wherein said identification module is further configured to initiate execution of a classification module based on the detection of said one or more seizure-related events;
a classification module including a processor configured to selectively execute a second group of one or more seizure-detection routines for classifying individual ones among said one or more seizure-related events as being associated with one or more physiological activity types, the one or more physiological activity types including a generalized tonic-clonic seizure type and at least one other physiological activity type; and
an alarm initiation module including a processor configured to send one or more alarms to said one or more caregiver devices in response to detection of said one or more seizure-related events.

23. The system of clause 22 wherein said one or more alarms may include one or more warning messages, one or more emergency alarms, or a combination of both.

24. The system of clause 22 wherein said identification module is calibrated to control a duty cycle of operation for at least one seizure detection routine among said second group of one or more seizure-detection routines.

25. The system of clause 24 wherein identification module is calibrated so that a duty cycle of at least one of said at least one seizure-detection routine among said second group of one or more seizure-detection routines operates so that a battery lifetime of the system is at least about 50% of a battery lifetime that may otherwise be achieved without execution of said at least one seizure-detection routine among said second group of one or more additional seizure-detection routines.

26. The system of clause 22 wherein said EMG detection system includes a battery having a lifetime of about 12 hours to about 36 hours.

27. A method of monitoring a patient for seizure activity comprising:
monitoring a patient using one or more EMG electrodes to obtain an EMG signal;
processing, with a processor, said EMG signal to determine if said patient may be experiencing one or more seizure-related events said processing including executing at least one of a first group of one or more first seizure-detection routines, said one or more first seizure-detection routines include instructions for calculating one or more property values of said EMG signal and comparing said one or more property values to one or more thresholds in detection of said one or more seizure-related events;
executing one or more second seizure-detection routines;
wherein said one or more second seizure-detection routines include instructions for classifying individual ones among said one or more seizure-related events in order to obtain classified seizure-related event data;
wherein said classified seizure-related event data include an identification of a relationship of said individual seizure-related events to one or more physiological activity types;
wherein said one or more physiological activity types include a generalized tonic-clonic seizure type and at least one other physiological activity type, said at least one other physiological activity type selected from a psychogenic non-epileptic seizure type, a non-seizure movement type, and a physiological activity type that includes a combination of both said psychogenic non-epileptic seizure type and said non-seizure movement type;
evaluating one or more performance metrics for said one or more seizure-detection routines when using said one or more thresholds with respect to at least one of said one or more physiological activity types; and
adjusting said one or more thresholds based on said one or more performance metrics.

28. The method of clause 27 wherein said one or more seizure-detection routines include at least one seizure-detection routine configured to determine one or more of a T-squared value and a principal component value.

29. The method of clause 27 wherein said one or more property values includes a T-squared value and said one or more thresholds includes a threshold T-squared value.

30. The method of clause 27 wherein said one or more property values includes a principal component value and said one or more thresholds includes a threshold principal component value.

31. The method of clause 27 wherein said one or more property values include a number of zero crossings exhibiting a hysteresis and said one or more thresholds include a threshold number of zero crossings.

32. The method of clause 27 wherein said one or more other seizure-detection routines includes at least one seizure-detection routine configured to classify a detected seizure-related event as said generalized tonic-clonic seizure type based on an identification of each of a tonic phase and a clonic phase in said EMG signal;
wherein said tonic phase is recognized if a scaled magnitude of a high frequency component of said EMG signal is greater than a high frequency threshold; and
wherein said clonic phase is recognized if a scaled magnitude of a lower frequency component of said EMG signal is greater than a lower frequency threshold.

33. The method of clause 27 wherein said one or more other seizure-detection routines includes at least one seizure-detection routine configured to classify a detected seizure-related event as a seizure including a clonic phase based on the detection of one or more qualified clonic-phase bursts, calculation of a burst activity level, and compare said burst activity level to one or more activity level thresholds.

34. The method of clause 27 further comprising selectively executing said one or more other seizure-detection routines in response to a detection of said one or more seizure-related events.

35. The method of clause 27 wherein said one or more performance metrics includes at least one sensitivity metric.

36. The method of clause 35 wherein said sensitivity metric is evaluated with respect to said generalized tonic-clonic seizure activity type.

37. The method of clause 35 wherein said at least one sensitivity metric is a patient-specific sensitivity.

38. The method of clause 35 wherein said at least one sensitivity metric is a group sensitivity.

39. The method of clause 35 wherein said at least one sensitivity metric includes a patient sensitivity and a group sensitivity and further comprising:
selection of either of said patient sensitivity or said group sensitivity based on whether a statistically significant patient-specific reference set of classified physiological events is available.

40. The method of clause 27 wherein said adjusting of said one or more thresholds applies an adjusted threshold setting wherein one or more sensitivity performance metrics are above a desired level and wherein one or more other performance metrics are adjusted to enhance battery performance.

41. The method of clause 27 wherein said one or more performance metrics are selected from a selectivity performance metric, a rate of detection performance metric, and a combination of said performance metrics.

42. A method of monitoring a patient for seizure activity comprising:
monitoring said patient using one or more EMG electrodes to obtain an EMG signal;
processing, with a processor, said EMG signal to determine if said patient may be experiencing a seizure-related event;
wherein said processing includes executing at least one of a first group of one or more seizure-detection routines;
wherein said one or more seizure-detection routines include instructions for calculating one or more property values for said EMG signal and comparing said one or more property values to one or more thresholds in detection of said seizure-related event;
executing one or more other seizure-detection routines;
wherein said one or more other seizure-detection routines include instructions for classifying said seizure-related event as being associated with one or more physiological activity types;
wherein said one or more physiological activity types include an epileptic seizure activity type and at least one other physiological activity type; and
executing an emergency alarm if said seizure-related event is classified as being of said epileptic seizure activity type.

43. The method of clause 42 further comprising:
queuing an emergency alarm for transmission when said seizure-related event is detected;
sending a caregiver classification data showing how said seizure-related event was classified; and
providing said caregiver with a mechanism to automatically cancel said emergency alarm.

44. The method of clause 42 further comprising:
initiating execution of a warning alarm when said seizure-related event is detected; sending a caregiver classification data showing how said seizure-related event was classified; and
providing said caregiver with a mechanism to automatically cancel said emergency alarm.

45. A method of monitoring a patient for seizure activity comprising:
monitoring said patient using one or more EMG electrodes to obtain an EMG signal;
processing, with a processor, said EMG signal to determine if said patient may be experiencing one or more seizure-related events;
wherein said processing includes executing at least one of a first group of one or more seizure-detection routines said one or more seizure-detection routines including instructions for calculating one or more property values of said EMG signal and comparing said one or more property values to one or more thresholds in detection of said one or more seizure-related events;
initiating execution of one or more other seizure-detection routines if said processing indicates that said patient has experienced at least one of said one or more seizure-related events, said one or more other seizure-detection routines including instructions for classifying individual ones among said one or more seizure-related events in order to create classified seizure-related event data; and
initiating one or more alarms if said classified seizure-related event data indicates that said patient has experienced a seizure.

46. The method of clause 45, wherein said one or more seizure-detection routines include at least one seizure-detection routine configured to determine either of a T-squared value or a principal component value.

47. The method of clause 45, said one or more other seizure-detection routines including at least one seizure-detection routine configured to classify a detected seizure-related event as a generalized tonic-clonic seizure type based on an identification of each of a tonic phase and a clonic phase in said EMG signal;
wherein said tonic phase is recognized if a scaled magnitude of a high frequency component of said EMG signal is greater than a high frequency threshold; and
wherein said clonic phase is recognized if a scaled magnitude of a lower frequency component of said EMG signal is greater than a lower frequency threshold.

48. An EMG detection system configured for automatic calibration of threshold settings for monitoring a patient for detection of seizure activity, the system comprising:
a wireless EMG detection unit (32), said wireless EMG detection unit including one or more EMG electrodes (54), the one or more EMG electrodes configured to collect an EMG signal from a patient, the wireless EMG detection unit (32) configured to remotely communicate with one or more caregiver devices (42, 44);
an identification module (92), said identification module including a processor configured to execute a first group of one or more first seizure-detection routines for determining one or more property values of said EMG signal and comparing said one or more property values to one or more initial thresholds for detection of one or more seizure-related events;
a classification module (94), said classification module including a processor configured to execute a second group of one or more second seizure-detection routines for classifying individual seizure-related events as being associated with one or more physiological activity types, the one or more physiological activity types including a generalized tonic-clonic seizure type and a non-seizure activity type; and
a threshold adjustment module (96), said threshold adjustment module including a processor configured to automatically adjust at least one of said one or more initial thresholds if a threshold number of said one or more seizure-related events are detected and classified as a non-seizure activity type.

49. The system of clause 48 wherein said threshold adjustment module is configured to evaluate one or more performance metrics for said one or more seizure-detection routines when the routines are applying said one or more initial thresholds or when the routines are applying one or more adjusted threshold settings, the one or more performance metrics including a sensitivity for detection of generalized tonic-clonic seizures and a selectivity for detection of generalized tonic-clonic seizures.

50. The system of clause 48 wherein said classification module is configured to further classify seizure-related events classified to be generalized tonic-clonic seizures based on a total duration of said generalized tonic-clonic seizure, a duration of the tonic phase of said generalized tonic-clonic seizure, and a duration time of the clonic phase of said generalized tonic-clonic seizure.

51. The system of clause 50 further configured to send classification data to a caregiver in real-time.

52. The system of clause 48 wherein said identification module is included in said wireless detection unit and said classification module is included in a base station in remote communication with said wireless detection unit;
wherein said wireless detection unit is configured to execute at least one of said first group of one or more first seizure-detection routines in a substantially continuous manner for at least about 24 hours;
wherein the device is calibrated for a patient so that a duty cycle of operation of said second group of one or more second seizure-detection routines is less than about 1:100.

53. The system of clause 52 wherein said at least one of said first group of one or more first seizure-detection routines is configured for determining a number of zero crossings exhibiting a hysteresis.

54. The system of clause 52 wherein said at least one of said first group of one or more first seizure-detection routines is configured for determining an amplitude of said EMG signal.

55. The system of clause 52 wherein said at least one of said first group of one or more first seizure-detection routines is configured for determining either of a T-squared statistical value or a principal component value.

Although the disclosed systems, methods, and apparatuses and their advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition, or matter, means, methods and steps described in the specification. Use of the word “include,” for example, should be interpreted as the word “comprising” would be, i.e., as open-ended. As one will readily appreciate from the disclosure, processes, machines, manufactures, compositions of matter, means, methods, or steps presently existing or later to be developed that perform substantially the same function or achieve substantially the same result as the corresponding embodiments described herein may be utilized. Accordingly, the appended claims are intended to include within their scope such processes, machines, manufactures, compositions of matter, means, methods or steps.

What is claimed is:

1-11. (canceled)

12. A method of calibrating an EMG system for monitoring a patient for seizure activity, the method comprising:
disposing an EMG detection unit including one or more EMG electrodes in association with one or more patient muscles, said one or more EMG electrodes configured for collecting an EMG signal in a form substantially representing seizure-related muscle activity;
collecting said EMG signal using said one or more EMG electrodes;
processing said EMG signal using a first group of one or more seizure-detection routines, the one or more seizure-detection routines configured for determining one or more property values of said EMG signal and comparing said one or more property values to one or more initial threshold in order to detect one or more seizure-related events;
classifying said one or more seizure-related events using a second group of one or more additional seizure-detection routines, said one or more additional seizure-detection routines configured to determine how individual seizure-related events relate to one or more physiological activity types, the one or more physiological activity types including a generalized tonic-clonic seizure type and at least one other physiological activity type;
evaluating how well said first group of one or more seizure-detection routines functions in detecting said one or more seizure-related events based on one or more performance metrics for said one or more seizure-detection routines when said one or more seizure-detection routines apply said one or more initial thresholds, the one or more performance metrics including a sensitivity for detection of generalized tonic-clonic seizures and a selectivity for detection of generalized tonic-clonic seizure; and
updating said one or more initial thresholds based on the evaluation of said one or more performance metrics in order to calibrate said EMG detection unit.

13. The method of claim 12 further comprising initiating one or more alarms based on the detection of said one or more seizure-related events.

14. The method of claim 12 wherein said sensitivity for detection of generalized tonic-clonic seizures comprises either of a group sensitivity for detection of generalized tonic clonic seizures or a patient-specific sensitivity for detection of generalized tonic-clonic seizures and further comprising:
selecting either of said group sensitivity for detection of generalized tonic-clonic seizures or said patient-specific sensitivity for detection of generalized tonic-clonic seizures based on a number of classified generalized tonic-clonic seizures for said patient.

15. The method of claim 12, said sensitivity for detection of generalized tonic-clonic seizures includes a weighted contribution of group sensitivity and patient-specific sensitivity.

16. The method of claim 12, said performance metrics further including how said first group of one or more seizure-detection routines impact a duty cycle of at least one seizure-detection routine among said second group of one or more additional seizure-detection routines.

17. The method of claim 16 wherein a maximum duty cycle of said at least one seizure-detection routine among said second group of one or more additional seizure-detection routines is selected so that a battery lifetime is at least about 50% of a battery lifetime that may otherwise be achieved without execution of said at least one seizure-detection routine among said second group of one or more additional seizure-detection routines.

18. (canceled)

19. (canceled)

20. The method of claim 12, said second group of one or more seizure-detection routines including one or more seizure-detection routine configured to execute a wavelet transform in order to organize EMG signal into a high frequency band of EMG signal and a low frequency band of EMG signal, and analyze said high frequency band of EMG signals and said low frequency band of EMG signals in order to determine whether a tonic phase and/or a clonic phase of a generalized tonic-clonic seizure is present.

21. (canceled)

22. An EMG detection system for monitoring of a patient for detection of seizure activity, the system comprising:
a wireless EMG detection unit, said wireless detection unit including one or more EMG electrodes, the one or more EMG electrodes configured to collect an EMG signal for a patient substantially continuously over time;
wherein said detection unit is configured for remote communication with one or more caregiver devices;
an identification module including a processor configured to execute a first group of one or more seizure-detection routines for determining one or more property values of said EMG signal and comparing said one or more property values to one or more initial thresholds for detection of one or more seizure-related events;
wherein said identification module is further configured to initiate execution of a classification module based on the detection of said one or more seizure-related events;
a classification module including a processor configured to selectively execute a second group of one or more seizure-detection routines for classifying individual ones among said one or more seizure-related events as being associated with one or more physiological activity types, the one or more physiological activity types including a generalized tonic-clonic seizure type and at least one other physiological activity type; and
an alarm initiation module including a processor configured to send one or more alarms to said one or more caregiver devices in response to detection of said one or more seizure-related events.

23. The system of claim 22 wherein said one or more alarms may include one or more warning messages, one or more emergency alarms, or a combination of both.

24. The system of claim 22 wherein said identification module is calibrated to control a duty cycle of operation for at least one seizure detection routine among said second group of one or more seizure-detection routines.

25. The system of claim 24 wherein identification module is calibrated so that a duty cycle of at least one of said at least one seizure-detection routine among said second group of one or more seizure-detection routines operates so that a battery lifetime of the system is at least about 50% of a battery lifetime that may otherwise be achieved without execution of said at least one seizure-detection routine among said second group of one or more additional seizure-detection routines.

26. The system of claim 22 wherein said EMG detection system includes a battery having a lifetime of about 12 hours to about 36 hours.

27-47. (canceled)

48. An EMG detection system configured for automatic calibration of threshold settings for monitoring a patient for detection of seizure activity, the system comprising:
a wireless EMG detection unit, said wireless EMG detection unit including one or more EMG electrodes, the one or more EMG electrodes configured to collect an EMG signal from a patient, the wireless EMG detection unit configured to remotely communicate with one or more caregiver devices;
an identification module, said identification module including a processor configured to execute a first group of one or more first seizure-detection routines for determining one or more property values of said EMG signal and comparing said one or more property values to one or more initial thresholds for detection of one or more seizure-related events;
a classification module, said classification module including a processor configured to execute a second group of one or more second seizure-detection routines for classifying individual seizure-related events as being associated with one or more physiological activity types, the one or more physiological activity types including an epileptic seizure activity type and a non-seizure activity type; and
a threshold adjustment module, said threshold adjustment module including a processor configured to automatically adjust at least one of said one or more initial thresholds if a threshold number of said one or more seizure-related events are detected and classified as a non-seizure activity type.

49. The system of claim 48 wherein said threshold adjustment module is configured to evaluate one or more performance metrics for said one or more seizure-detection routines the one or more performance metrics including a sensitivity for detection of epileptic seizures and a selectivity for detection of epileptic seizures.

50. The system of claim 48 wherein said classification module is configured to further classify seizure-related events classified to be generalized tonic-clonic seizures based on a total duration of said generalized tonic-clonic seizure, a duration of the tonic phase of said generalized tonic-clonic seizure, and a duration time of the clonic phase of said generalized tonic-clonic seizure.

51. The system of claim 50 further configured to send classification data to a caregiver in real-time.

52. The system of claim 48 wherein said identification module is included in said wireless detection unit and said classification module is included in a base station in remote communication with said wireless detection unit;
wherein said wireless detection unit is configured to execute at least one of said first group of one or more first seizure-detection routines in a substantially continuous manner for at least about 24 hours;
wherein the device is calibrated for a patient so that a duty cycle of operation of said second group of one or more second seizure-detection routines is less than about 1:100.

53. The system of claim 52 wherein said at least one of said first group of one or more first seizure-detection routines is configured for determining a number of zero crossings exhibiting a hysteresis.

54. (canceled)

55. The system of claim 52 wherein said at least one of said first group of one or more first seizure-detection routines is configured for determining either of a T-squared statistical value or a principal component value.
